(19) United States
(12) Patent Application Publication
(10) Pub. No.: US 2016/0213317 A1
(43) Pub. Date: Jul. 28, 2016

(54) WIRELESS NON-INVASIVE ANIMAL MONITORING SYSTEM AND RELATED METHOD THEREOF

(71) Applicants: Neal T. RICHARDSON, Charlottesville, VA (US); Aaron O. OLOWIN, Charlottesville, VA (US); Rebecca A. BIRD, Earlysville, VA (US); Brian R. CLARK, Charlottesville, VA (US); Patricia L. FOLEY, Arlington, VA (US); Eugene B. PARKER, JR., Charlottesville, VA (US); University of Virginia Patent Foundation, Charlottesville, VA (US)

(72) Inventors: Neal T. RICHARDSON, Charlottesville, VA (US); Aaron O. OLOWIN, Charlottesville, VA (US); Rebecca A. BIRD, Earlysville, VA (US); Patricia L. FOLEY, Arlington, VA (US); Eugene B. PARKER, JR., Charlottesville, VA (US)

(21) Appl. No.: 15/025,547

(22) PCT Filed: Sep. 30, 2014
(86) PCT No.: PCT/US2014/058413

§ 371 Date: Mar. 28, 2016

Related U.S. Application Data
(60) Provisional application No. 61/884,308, filed on Sep. 30, 2013.

(51) Int. Cl. A61B 5/00; A61B 5/0205; A61B 5/113; A61B 5/0476; A61B 5/145; A61B 6/00; A61B 5/0402

(52) U.S. Cl. CPC A61B 5/6804; A61B 5/0059; A61B 6/508; A61B 5/02055; A61B 5/0402; A61B 5/0476; A61B 5/14542; A61B 5/1135; A61B 5/7278; A61B 5/0002; A61B 2503/40; A61B 5/024

(57) ABSTRACT

A system, associated method, and computer readable medium for monitoring one or more test subjects. The system is comprised of a subject wearable interface which features at least one sensor to read data from the test subject. A processor module receives data from the sensor(s) on the subject wearable interface. The system includes an output module and a wireless transceiver module that receive data from the processor module, any of which may or may not be located on the subject wearable interface. The system allows for the subject to be monitored while wearing the subject wearable interface without being tethered to any sensing device or data acquisition device that would be wired and restrain the subject from normal movement. The system allows a researcher or user (or device) to record data on a test subject while the test subject is allowed to freely move and engage in normal activity.

### CROSS-REFERENCE TO RELATED APPLICATIONS

The present application claims benefit of priority under 35 U.S.C. §119(e) from U.S. Provisional Application Ser. No. 61/884,308, filed Sep. 30, 2013, entitled “Wireless Non-Invasive Animal Monitoring System and Related Method thereof;” the disclosure of which is hereby incorporated by reference herein in its entirety.

### STATEMENT OF GOVERNMENT INTEREST

This invention was made with government support under Grant Nos. R43RR024944-01 and R44RR024944-03 awarded by the National Institutes of Health. The government has certain rights in the invention.

### FIELD OF THE INVENTION

The present invention relates generally to the field of non-invasive animal monitoring. More specifically, the invention is in the subfield of a wireless small animal physiological monitoring system configured to be used with wearable interface worn by a subject.

### BACKGROUND

Monitoring test subjects during experiments or studies poses a number of challenges for researchers. It is often desirable to acquire and record data from the test subjects as they undergo testing, whether the study involves results that are immediate or that occur over longer time intervals. However, traditional methods for recording data from the test subjects involve the use of electronic sensors that require wires or surgical implantation of radiotelemetric sensors. The use of wired data acquisition on living test subjects necessitates that the subjects either be totally incapacitated, for example with anesthesia or restraints, or endure a serious hindrance of movement. If the subject is allowed to move about, it can compromise the quality and quantity of useful data that can be obtained or recorded. Using existing prior art systems, if the subject is not restrained, it will often move about in a way that pulls at the wires that are connected to the subject. This results in sensors being pulled away from the subject, either partially or completely, and degrading the quality and quantity of data recorded. It is also problematic that the use of wired monitoring could cause the subject to damage or destroy the equipment, potentially injuring the subject and requiring replacement of costly equipment. The constant attachment of a wired monitoring system may also invoke a stress response in the subject, either from restricted movement or because the subject may not behave as normal, influencing the outcome of an experiment. Because of these issues, wired monitoring of test subjects cannot be used continuously throughout a study, and severely limits the amount of data that can be collected. Radiotelemetry can be used to capture high-quality data but requires an invasive surgical procedure which is stressful and potentially painful to the subject. It is further limited by the ability to capture data from only one subject per enclosure.

### Overview

An aspect of an embodiment of the present invention provides, among other things, a wireless non-invasive animal monitoring system and related method. In an embodiment, for example, the system and related method provides, among other things, the capability to monitor up to eighteen animals simultaneously, with continuous recording, synchronization, and display of physiological data, including heart rate, respiratory rate, motion activity, skin temperature, and ambient temperature; as well as other possible parameters and characteristics as required, needed or desired. It should be appreciated that more or less than eighteen animals or subjects may be simultaneously monitored. In addition to continuous recording, the system records data intermittently, thus greatly increasing the battery life of the remote units mounted on the animals. Data may be collected wirelessly via one remote unit per animal (or other ratio as desired, needed or required) by a base station that is in communication with (hard wired or wireless) a PC and/or other processor, system or network that may be wireless or with wire/hardware. Each remote unit may be wired into sensors contained within a miniature, lightweight jacket worn just behind the front legs of each animal. Wireless cameras may be integrated into the system for remote visual monitoring of animals in real time or to capture views from the subject's perspective. The remote units may be in wireless communication with the sensors that are contained within or in communication with a miniature jacket (or belt, band, strap or other attire) that may be worn or disposed behind or in adjacent to the front legs of each animal (subject). It should be appreciated that wireless features may be replaced with hard wired components; however, in doing so the mobility characteristics associated with the system and other advantages may be compromised.

An aspect of an embodiment of the present invention provides, but not limited thereto, a system for monitoring a subject. The system may comprise: at least one sensor module disposed in communication with the subject and configured to obtain data from the subject; at least one processor module configured to receive the subject data; a transmission module or transceiver module configured to transmit the subject data, wherein the transmission comprises wireless transmission to an output module that is remote relative to the subject; and wherein at least one of the at least one sensor module, the at least one processor module, or the transmission module is configured to be disposed in communication with a subject wearable interface.

An aspect of an embodiment of the present invention provides, but not limited thereto, a method for monitoring a subject. The method may comprise: providing a subject wearable interface; disposing at least one sensor module in communication with the subject, wherein the at least one sensor module is configured to be in communication with the subject wearable interface; obtaining data from the subject using the at least one sensor module; and transmitting the subject data to an output module that is remote relative to the subject.

An aspect of an embodiment of the present invention provides, but not limited thereto, a non-transitory machine readable medium including instructions, which when executed by a machine, cause the machine to: obtain data from the subject using at least one sensor module; and transmit the subject data to an output module that is remote relative to the subject.

An aspect of an embodiment of the present invention provides, but not limited thereto, a system, associated method, and computer readable medium for monitoring one or more test subjects. The system is comprised of a subject wearable interface which features at least one sensor to read data from the test subject. A processor module receives data from the sensor(s) on the subject wearable interface. The system also includes an output module and a wireless transceiver module that receive data from the processor module, any of which may or may not be located on the subject wearable interface. The system allows for the subject to be monitored while wearing the subject wearable interface without being tethered to any sensing device or data acquisition device that would be wired and restrain the subject from normal movement. The system allows a researcher or user (or applicable device or system) to record data on a test subject while the test subject is allowed to freely move and engage in normal activity.

These and other objects, along with advantages and features of various aspects of embodiments of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 shows a schematic block diagram of an embodiment of the present invention small animal monitoring system.

FIG. 2 shows a schematic block diagram of an embodiment of the present invention small animal monitoring system.

FIG. 3 shows a schematic block diagram of an embodiment of the present invention small animal monitoring system.

FIG. 4 is a block diagram illustrating an example of a machine upon which one or more aspects of embodiments of the present invention can be implemented.

FIG. 5 provides a photographic depiction of an embodiment of the rat jacket and mouse jacket and associated battery and circuit board, whereby the jackets are illustrated in a laid open position without the subject present.

FIGS. 6 and 7 each illustrate an example of the graphical users interface (GUI) associated with an embodiment of the present invention.

FIG. 8 illustrates three rats that were allowed to move freely in a container while instrumented using an embodiment of the present invention RSM system.

FIG. 9 illustrates two mice that were allowed to move freely in a container while instrumented using an embodiment of the present invention RSM system.

FIGS. 10-13 graphically depict example time series data streams collected using an embodiment of the present invention wireless SAM system on animals. FIG. 10 provides the example activity data. FIG. 11 provides the example Electrocardiography (ECG) data. FIG. 12 provides the example respiratory band data. FIG. 13 provides the example skin temperature data.

FIG. 14 graphically summarizes the HR measurement discrepancies between the SAM and MouseOx systems for rats and mice.

FIG. 15 graphically summarizes the RR measurement discrepancies between the SAM and MouseOx systems for rats and mice.

FIG. 16 provides a perspective schematic view of either mouse or rat wearing a remote sensor module and related components.

FIG. 17 provides a plan schematic view of either mouse or rat wearing a remote sensor module and related components.

FIG. 18 provides a perspective schematic view of an aspect of an embodiment the present invention small animal monitoring system.

FIG. 19 is an illustrative screenshot of an embodiment of the SAM desktop software application.

FIG. 20 is an illustrative screenshot of an embodiment of the SAM desktop software application.

### DETAILED DESCRIPTION OF EXEMPLARY EMBODIMENTS OF THE PRESENT INVENTION

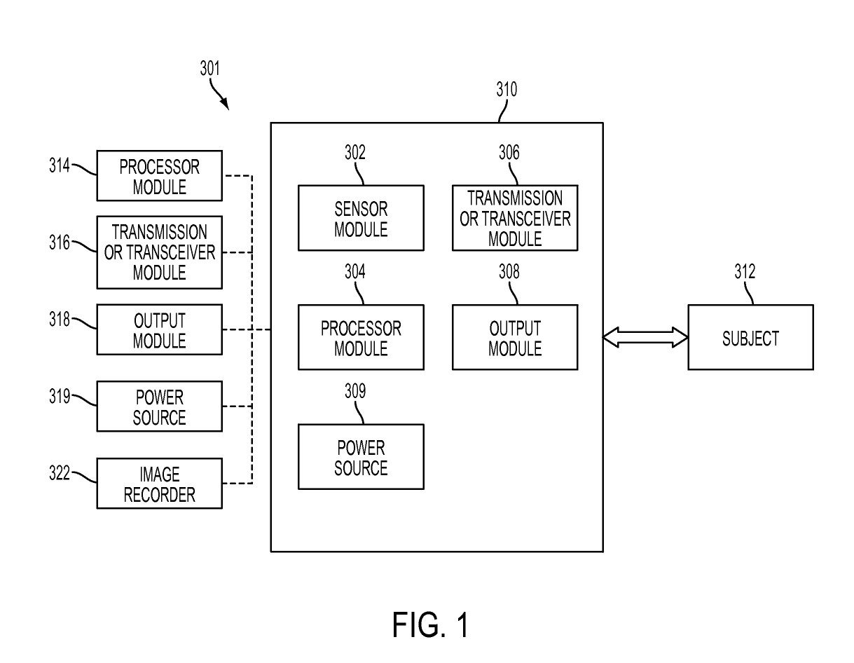

FIG. 1 provides an aspect of an embodiment of the present invention monitoring system 301 comprising at least one sensor module 302 disposed in communication with a subject 312, such as a rodent or the like. The sensor module 302 may be configured to obtain one or more of any combination of the following physiological related data: heart rate (HR) data, respiratory rate (RR) data, Electrocardiography (ECG) data, Electroencephalography (EEG) data, arterial oxygen saturation (SaO2), photoplethysmography data, temperature data, chest contraction and expansion data, or other physiological data as required, desired, or needed. For example, the sensor module 302 may be configured to obtain one or more of any combination of the following subject data: heart rate (HR) data; chest contraction and expansion data, acceleration due to subject movement and/or gravitational forces imposed on the subject 312 (i.e., body posture/orientation), and temperature data at the subject 312. It should be appreciated that an image recording systems, including cameras, may be used to collect physiological or other data from the subject. The image recording system or camera may be configured to detect visible wavelengths, infrared wavelengths, ultraviolet wavelengths, X-ray wavelengths, or any other wavelength as desired, required or needed for any particular application. A processor module 304 may be provided to receive the subject data and may optionally be disposed in communication with the subject 312 whereby the processor module 304 may be configured to 1) measure an ECG signal from which may be computed the heart rate (HR); (2) compute respiratory rate (RR) which may be derived from the chest contraction and expansion data; (3) compute movements of the animal which may be derived from the acceleration data; (4) skin temperature (ST) derived from the temperature data at the subject; (5) computing HR from photoplethysmography data or sensor; and (6) inferring respiration from a HR signal (respiratory sinus arrhythmia). A transmission or transceiver module 306 may optionally be disposed in communication with the subject 312 to transmit subject data and information derived by the sensor module 302 and/or processing module 304. It should be appreciated that the system or components may have the ability to both transmit and receive, such as if any of the subject wearable interface 310 related parameters (e.g., sampling rate) are set by the PC or other remote processor. The transmission or transceiver module 306 may be wireless, hardwired, or a combination of wireless and hardwired. An output module 308 may optionally be disposed in communication with the subject 312 to receive transmitted data and information derived by the sensor module 302 and/or processing module 304. The output module 308 includes, for example, storage, memory, network, or a display. A power source 314 may be provided in communication with any combination of one or more of the following; sensor module 302, processor module 304, transmission or transceiver module 306, or output module 308. Moreover, any combination of one or more of the following; sensor module 302, processor module 304, transmission or transceiver module 306, output module 308, and power source 314, may be in disposed in communication with the subject wearable interface 310. The wearable interface 310 may include a variety of structures or mechanism to accommodate or retain the various aforementioned modules or components in place upon the subject. For example, the wearable interface 310 that contains or retains the various modules or components may include a jacket, strap, sleeve, wrap, drape, cuff, band, vest, backpack, or the like as desire, required or needed. The wearable interface 310 may include various attachment or retention devices, structures, or materials for holding the various aforementioned modules and components in place. Optionally, an image recording device 322, such as a camera, video recorder or the like, can be in communication with the system 301 or components thereof to enable remote observation of experiments of subject 312, monitoring of the subject 312, and/or diagnoses of the subject 312.

Referring to FIG. 1, an aspect of an embodiment provides a system 301 for monitoring a subject 312 is shown. The system 301 is made up of at least one sensor module 302 disposed in communication with the subject 312 and configured to obtain data from the subject 312. The system 301 may include at least one processor module 304 or 314 configured to receive data from the subject 312. A transmission or transceiver module 306 or 316 may be configured to transmit the subject data wirelessly to an output module 318 that is remote relative to the subject 312. It should be appreciated that at least one of the sensor module 302, processor module 304, or transmission or transceiver module 306 may be configured to be disposed in communication with a subject wearable interface 310.

Still referring to FIG. 1, it should be appreciated that any number of different configurations may be used as desired or required to achieve specific functionality or meet particular requirements. For example, the system 301 may include a power source 309, which may be mounted in communication with the subject wearable interface 310. The system 301 may also be configured to include a power source 319 that is located remotely from the subject wearable interface 310, or a combination of a remote power source 319 and a power source 309 in communication with the subject wearable interface 310 is possible. In either case, the system 301 may still be in communication with the power source 309 or 319.

It should be appreciated that the system 301 can be configured to acquire physiological data from the subject 312 such as: heart rate (HR) data, respiratory rate (RR) data, ECG data, EEG data, arterial oxygen saturation (SaO2), photoplethysmography data, temperature data, chest contraction and expansion data, or other physiological data as required, desired, or needed. It should be appreciated that the system 301 can be configured to acquire physiological data from the subject 312, which may be a rodent. For example, the data acquired or recorded from the subject 312 may be one or more of any combination of the following: heart rate (HR) data, chest contraction and expansion data, acceleration due to subject movement and/or gravitational forces imposed on the subject 312 (i.e., body posture/orientation, and temperature data at or from the subject 312. It should be appreciated that the system 301 may include image recording systems, including cameras, may be used to collect physiological or other data from the subject. The image recording system or camera may be configured to detect visible wavelengths, infrared wavelengths, ultraviolet wavelengths, X-ray wavelengths, or any other wavelength as desired, required or needed for any particular application. For example, it should also be appreciated that the system 301 may have a processing module 304 or 314 configured to 1) measure an ECG signal from which may be computed the heart rate (HR); (2) compute respiratory rate (RR) which may be derived from the chest contraction and expansion data; (3) compute movements of the animal which may be derived from the acceleration data; (4) skin temperature (ST) derived from the temperature data at the subject; (5) computing HR from photoplethysmography data or sensor; and (6) inferring respiration from a HR signal (respiratory sinus arrhythmia) or any other data as desired, required, or needed. Any one or combination of these functions may be incorporated into the processor module 304 or 314.

Still referring to FIG. 1, the subject wearable interface 310 may be configured for accommodating one or more of any combination of the following: at least one sensor module 302, a processing module 304, a transmission or transceiver module 306, or a power source 309. It should be appreciated that the system 301 may also be in communication with another power source 319.

The system 301 may contain a remote output module 318 that includes storage, memory, network, or display, or any combination thereof. Furthermore, the system 301 may be configured to allow for hard wired transmission or wireless transmission to an output module 308 that is local to the subject 312. It should be appreciated that this local output module 308 may be configured to be disposed in communication with the subject wearable interface 310. This local output module 308 may include storage, memory, or any combination thereof.

Referring generally to FIG. 1, it should be appreciated that remote is generally defined as providing a variable or constant distance away from the subject 312 so as to be separated from the subject 312. In contrast, local is generally understood to be generally in direct contact with subject 312 or adjacent thereto. It should be appreciated that any one of the modules shown may be disposed remotely or locally with respect to the subject 312.

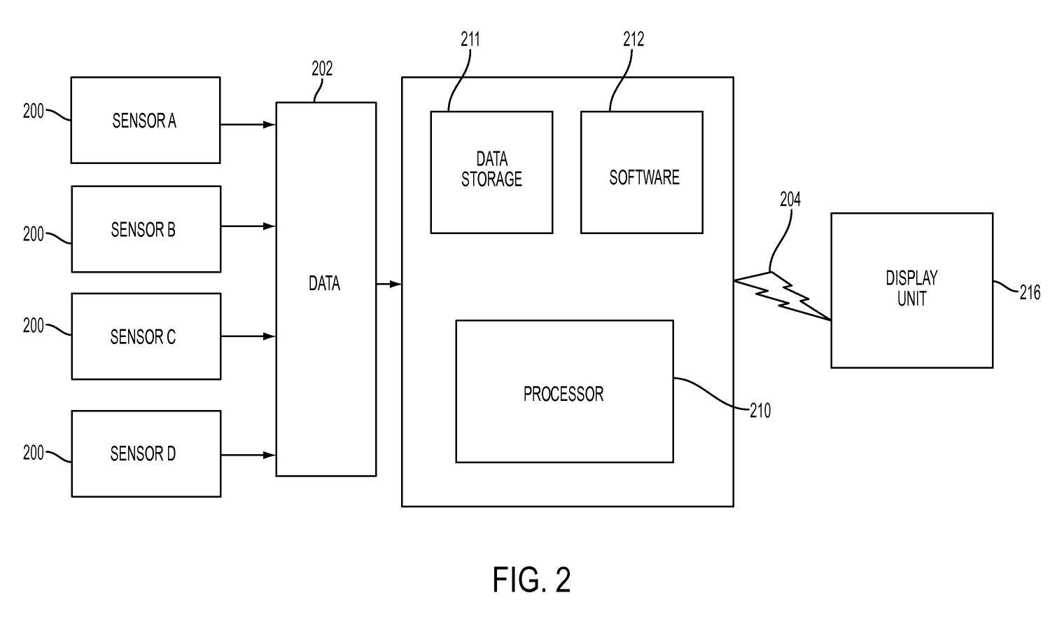

FIG. 2 depicts a high-level schematic diagram of an aspect of embodiment of the present invention. Sensor modules 200 provide data input 202, such as heart rate (HR) data, ECG, EEG, or photoplethysmography data, chest contraction and extraction data, acceleration due to subject movement and/or gravitational forces imposed on the subject, temperature data, or other physiological data, as well as other data as desired, needed or required, where the data is stored by the data storage unit 211, which can be either contained within the processor 210 and/or remotely. The data is then processed by the processor 210 using the software 212 and transmitted via a communication channel 204 either wirelessly or hard-wired (or combination thereof) to the display unit 216. It should be appreciated that a communication channel may be implemented between any of the modules (components) displayed in FIG. 2, as well as modules (or components) discussed throughout this disclosure.

It should be appreciated that any of the components or modules referred to for the present invention embodiments as discussed in FIG. 2 (as well as embodiments throughout this disclosure, including the references incorporated by reference herein), may be integrally or separately formed with one another and implemented accordingly for the practicing the invention. Further, redundant functions or structures of the components or modules may be implemented. Moreover, the various components may be communicated locally and/or remotely with any user/occupant/system/computer/processor. Moreover, the various components may be in communication via wireless and/or hardwire or other desirable and available communication means, systems and hardware.

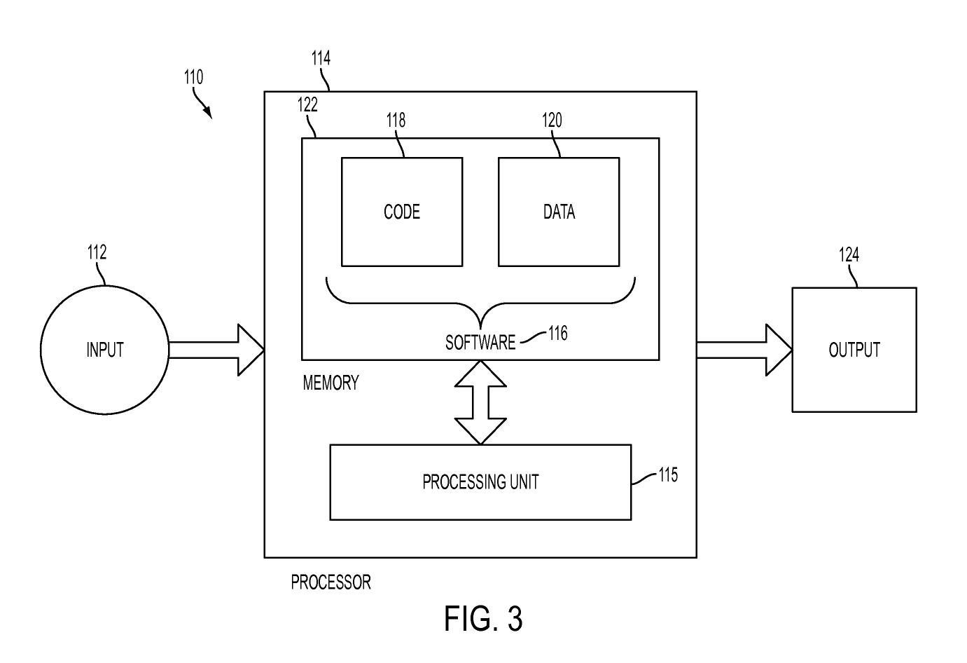

An aspect of an embodiment of the present invention provides, but not limited thereto, a system for monitoring a subject, such as a small animal like a rodent. Referring to FIG. 3, provided is a schematic of an approach of the small animal monitoring system 110 to be applied for obtaining physiological data or other data as provided by way of input module 112, for example. By way of the input module 112, subject-related data is received by the processor 114. Via the input module 112, subject related data is provided to the processor 114, wherein, the subject related data may include physiological related (or other data), such as heart rate (HR) data, ECG, EEG, or photoplethysmography data, chest contraction and expansion data, acceleration due to subject movement and/or gravitational forces imposed by the subject, and temperature data at the subject. The processing unit 115, for example, is in communication with a memory module 122. Next, an algorithm as represented by the software module 116, having code 118 and data 120 (i.e., software data), is configured to apply the related monitoring, observations, assessment, and diagnostic techniques and methods disclosed herein. A storage device is provided for, among other things, storing the software code and software data by way of the memory module 122, or a secondary memory module (not shown), as well as a combination of both of or additional memories. Alternatively, or in addition to the aforementioned memories, an output module 124 may be provided for outputting physiological information such as: heart rate (HR) data, respiratory rate (RR) data, ECG data, EEG data, arterial oxygen saturation (SaO2), photoplethysmography data, temperature data, chest contraction and expansion data, or other physiological data as required, desired, or needed. As an example, the output module 124 may be provided for outputting physiological information such as: 1) measure an ECG signal from which may be computed the heart rate (HR); (2) compute respiratory rate (RR) which may be derived from the chest contraction and expansion data; (3) compute movements of the animal which may be derived from the acceleration data; (4) skin temperature (ST) derived from the temperature data at the subject; (5) computing HR from photoplethysmography data or sensor; and (6) inferring respiration from a HR signal (respiratory sinus arrhythmia) or any other data as desired, required or needed.

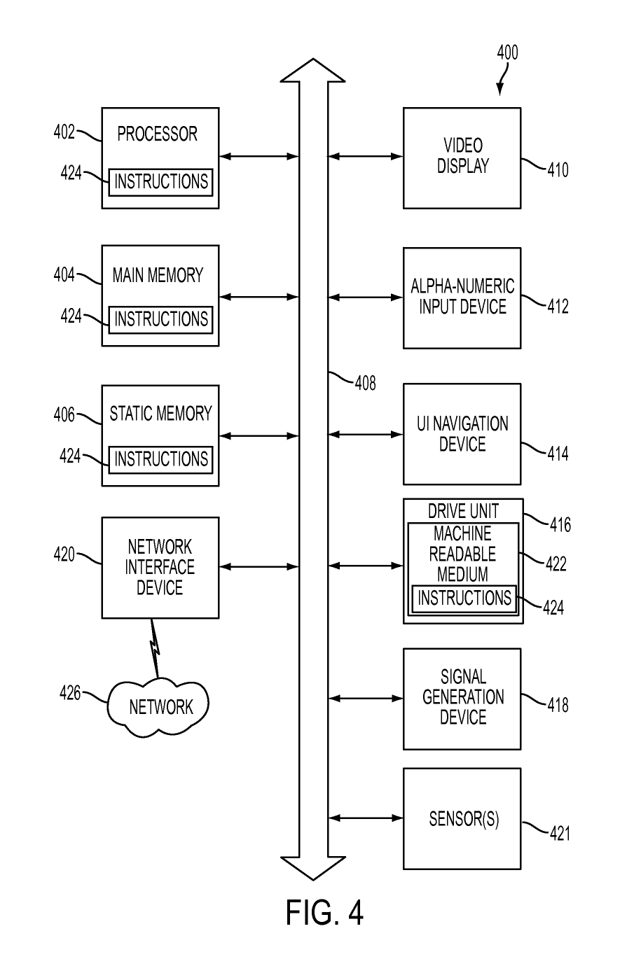

FIG. 4 illustrates a block diagram of an example machine 400 upon which one or more embodiments (e.g., discussed methodologies) can be implemented (e.g., run or executed).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or more processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a standalone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines. In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

An aspect of an embodiment of present invention provides a wireless Small Animal Physiological Monitor (SAM). An aspect of an embodiment of the present invention SAM system will provide, among other things, the capability to monitor up to 18 or more laboratory animals (for example, rats and mice) simultaneously, with continuous recording, synchronization, and display of physiological data, including heart rate, respiratory rate, motion activity, skin temperature, and ambient temperature. Data will be collected from each animal via a miniature, lightweight “backpack” that is worn on the dorsal surface of the animal, with sensors integrated into an appropriately-sized, chew-resistant design. Optional wireless cameras (sensitive to any wavelengths desired by the researcher or other use) can be mounted in laboratories or animal housing areas and integrated into the SAM system for remote visual monitoring of animals in real time. The subject wearable interface 310 or related components could also include a wireless camera to capture the perspective from the subject's vantage point. SAM system data (both physiological and video data) will be uploaded wirelessly to a shared receiver unit that interfaces to a PC (which can be local or over the Internet). A PC (or other processor or machine) will consolidate the incoming data stream(s), archive all data on a hard disk, and provide real-time data display, enabling remote, simultaneous monitoring of all instrumented animals. An aspect of an embodiment of the present invention SAM system will, among other things, significantly refine animal welfare for many research experiments by eliminating the need for surgical implantation of sensors and providing the capability for wireless monitoring of key physiological variables.

An aspect of an embodiment of the present invention SAM system will provide, but not limited thereto, improved instrumentation that will facilitate biomedical and behavioral science investigations across essentially all branches of the National Institutes of Health (NIH) (for example), as rats and mice are the most commonly used vertebrates in research. Additionally, an aspect of an embodiment the SAM system will enable research in areas that are presently constrained by the absence of suitable monitoring devices. For example, many questions regarding housing and husbandry practices of rodents and the impact of environmental conditions and/or external events on these animals remain unanswered.

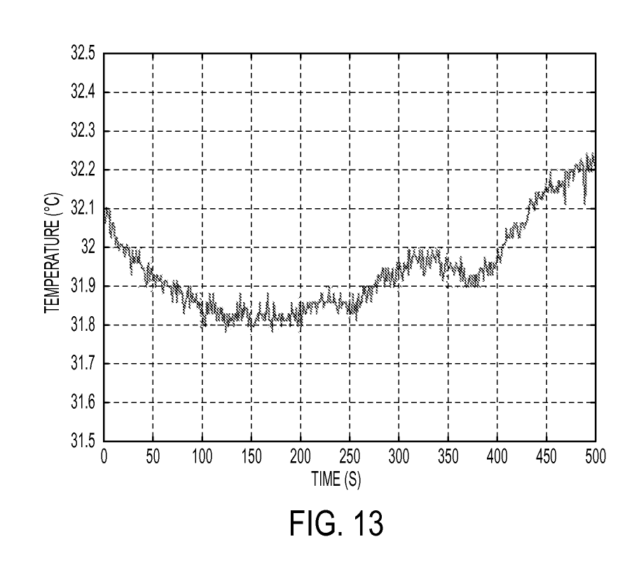

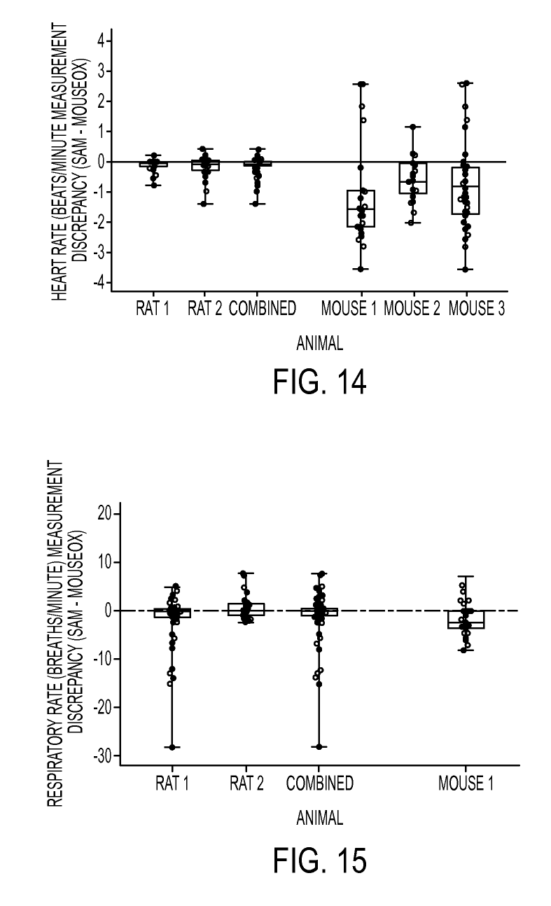

An aspect of an embodiment provides a wireless Small Animal Physiological Monitor (SAM) system that may provide, but not limited thereto, the capability to monitor up to 18 or more laboratory animals (e.g., rats and mice or the like) simultaneously, with continuous recording, synchronization, and display of physiological data. These data include heart rate (HR), respiratory rate (RR), three degrees-of-freedom (3-DOF) motion activity (ACT), skin temperature (ST), and ambient temperature (AT). Data will be collected from each animal via a miniature, lightweight remote sensing module (RSM) 618 and related components worn on the dorsal surface of the animal (e.g., rat 612 or mouse 613), as shown in FIGS. 16 and 17. In addition to physiological data, an aspect of an embodiment of the present invention SAM system can optionally include one or more wireless videographic cameras or still cameras that may be used to remotely monitor the animals from a PC or other processor based system.

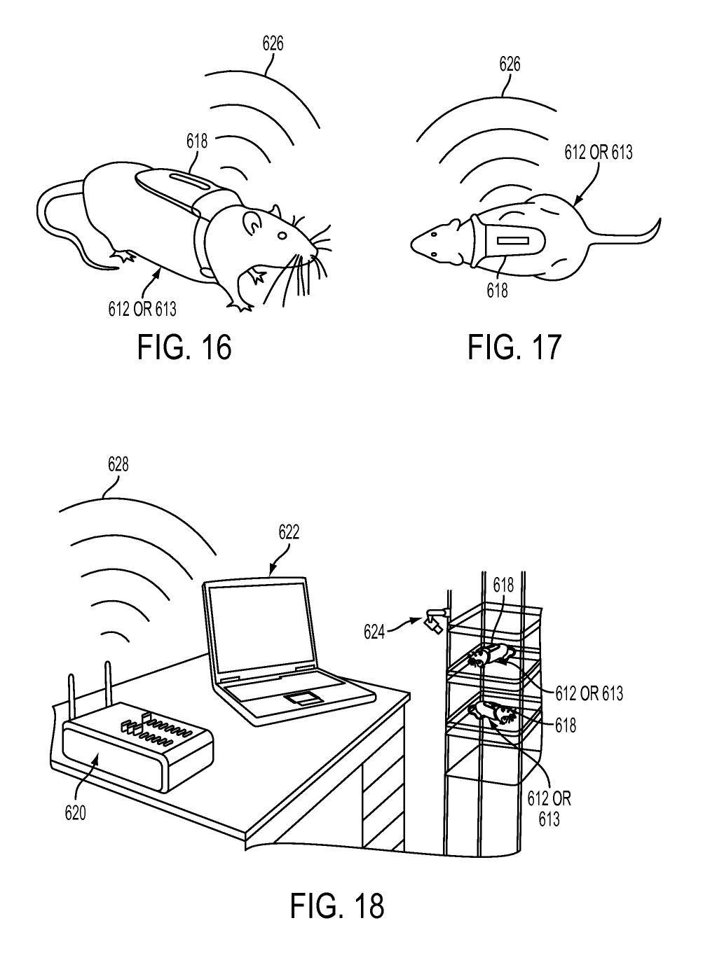

In an aspect of an embodiment, the data collected by the RSMs 618 may be uploaded wirelessly 626 (See FIGS. 16-17) to a system interface unit (SIU) 620 that will, in turn, may connect wirelessly 628 to a PC 622 or other processor or machine, as shown in FIG. 18. The SIU 620 will coordinate the incoming data streams from one or more animals (including video, if available) and send the data via the wireless IEEE 802.11 (i.e., “Wi-Fi”) protocol to the PC 622 for processing, displaying, and archiving. The SIU 620 will also serve as a docking station recharger for the lithium polymer battery or batteries that will power each RSM 618.

In an approach, custom SAM system software running on the PC may process the raw sensor data (e.g., to obtain HR from the ECG recording), display parameters, graphs, and/or video in real time for multiple animals, including any subset of monitored animals, simultaneously on screen, and archive the received data on a hard disk (including the video stream). Data transmissions from each RSM will be controllable using the PC software, enabling data to be collected intermittently (such as during a research experiment or daily checkup) or continuously. Furthermore, as the SIU may use a network communication protocol to interface with the PC, the PC can be any computer or processor (or system or device) with either a local or Internet connection, allowing for remote data access.

In an approach, a single RSM battery charge may last up to 24 hours or other predetermined period. The RSM battery charge can be replaced with a recharged battery without removing the backpack sensor system from the animal, enabling nearly continuous monitoring over days or weeks, or other predetermined duration. Moreover, in an approach, inductive charging (also known as “wireless charging”) may be implemented. For example, in an approach such inductive charging uses an electromagnetic field to transfer energy between two objects. This is usually done with a charging station. Energy is sent through an inductive coupling to an electrical device, which can then use that energy to charge batteries or run the device.

### EXAMPLES

Practice of an aspect of an embodiment (or embodiments) of the invention will be still more fully understood from the following examples and experimental results, which are presented herein for illustration only and should not be construed as limiting the invention in any way.

### Experimental Results and Examples Set No. 1

### Task 1: Develop SAM System Hardware and Software

An aspect of an embodiment of the wireless small animal monitor (SAM) system may be comprised of, but not limited thereto, two primary components: (1) up to 18 Remote Sensor Modules (RSMs), which are the data acquisition/transmission units that are worn by the animals; and (2) a System Interface Unit (SIU), which may consist of a Bluegiga iWRAP Access Server and the SAM Graphical User Interface (GUI) software application. Optionally, a wireless internet-enabled camera can be used with the SAM system to enable remote observation of experiments. The following paragraphs describe other possible components of an embodiment of the SAM system:

### Remote Sensor Modules (RSMs)

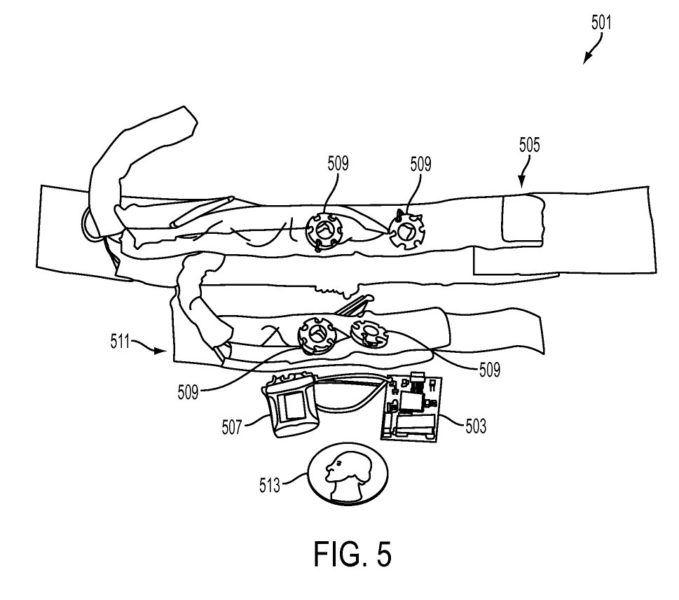

Referring to FIG. 5, in an embodiment, the RSMs modules may consist of: (1) a circuit board 503; (2) a battery 507; and (3) a wearable “jacket” form factor 505 or 511. The circuit board 503 may contain the circuitry required to, for example but not limited thereto, measure: (1) an ECG signal, which is used to compute heart rate (HR); (2) chest contraction and expansion, which are used to compute respiratory rate (RR); (3) the movements of the animal; and (4) skin temperature (ST). The circuit board 503 may consist of, for example but not limited thereto, the following components: microcontroller, accelerometer, and wireless transceiver.

### Microcontroller

In an embodiment, the Texas Instruments MSP430F2618 16-bit ultra-low-power microcontroller may be used to handle the sampling of analog data (e.g., ECG), the online calculation of periodic values such as the HR and RR (note that offline calculations can also performed), and storage of data prior to wireless transmission to the SIU. The microcontroller may have 116 KB of nonvolatile flash ROM (program memory) and 8 KB of volatile RAM (data memory).

### Accelerometer

In an embodiment, the Analog Devices ADXL345 3-axis accelerometer may be used to measure animals' activity, motion, and posture (i.e., body orientation). The ADXL345 can be set to any one of four different sensitivities by the microcontroller (±2 g, ±4 g, ±8 g, or ±16 g). For an aspect of an embodiment of the SAM system, sensitivity is set to ±2 g, as rat and mouse movements may generally lie in this range. At this level of sensitivity, the ADXL345 provides 0.004 g of resolution.

### Wireless Transceiver

In an embodiment, the RMS uses the Panasonic PAN1321-SPP class 2 Bluetooth module to transmit data between the RSM and the SIU. The PAN1321-SPP includes an antenna and contains the Bluetooth stack required for data transmission. As discussed in Task 2 (below, for example), the PAN1321-SPP module did not perform up to its published specifications; other versions of the SAM system will incorporate a different wireless transceiver.

### Battery

In an embodiment, the rechargeable, lithium polymer 65 mAh Flyzone FLZA6156 battery powers the RSM, which connects to a daughterboard via strong miniature magnets; the daughterboard may be hardwired directly to the main circuit board. The use of magnet connectors assures a firm and consistent connection between the battery and the circuitry, and facilitates quick battery changes (battery changes can be performed without removing the RSM from the animal).

### Jacket Form Factor

In an embodiment, the RSM jacket 505 encloses the circuit board 503 and battery 507, and holds the sensors that measure the animal's physiologic parameters in their proper position on the animal. As shown in FIG. 5, the jacket 505 may have snaps 509 to connect to the ECG electrodes, two stretchable sensors whose electrical resistance changes as the animals' chest and/or abdominal circumferences change (e.g., due to respiration), and a thermistor (to measure skin temperature). The accelerometers that measure activity, motion, and posture may be integrated into the RSM circuit board 503. Different size jackets may be implemented, for example, such as a rat jacket 505 or mouse jacket 511.

In an embodiment, to don the RSM, the ECG electrodes may be snapped into the jacket and then placed on the (usually anesthetized) animal. The jacket's Velcro straps may be attached to each other to encircle the animal (subject). The circuit board may then be connected to the wiring harness of the jacket (via an Omnetics nano-connector), and the battery may be attached to the circuit board (via the aforementioned magnets), and further secured to the jacket via hook and loop fastener (e.g., Velcro).

### System Interface Unit (SIU)

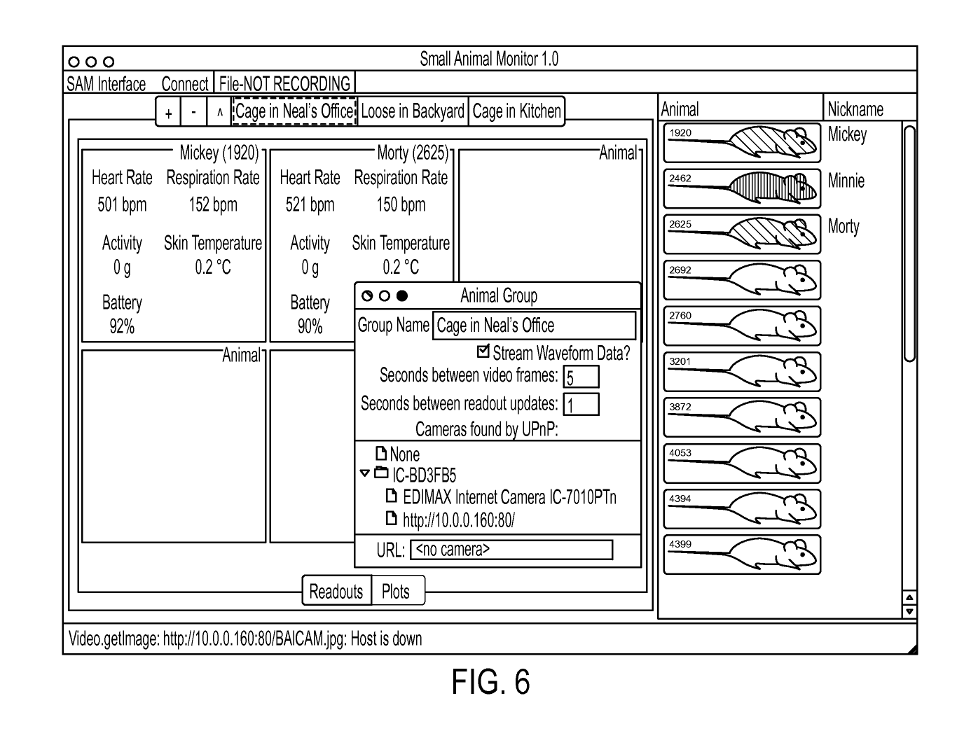

In an embodiment, the SAM GUI application (FIGS. 6 and 7) was developed in Java with Eclipse version 3.7.2 and runs on any operating system that supports a Java Virtual Machine. The application was tested with Java 1.6 on Mac OS X 10.6, Windows 7, and Ubuntu 11.4. For example, FIG. 6 provides a screenshot of an embodiment of the Sam GUI illustrating session configuration. For example, FIG. 7 provides a screenshot of an embodiment of the Sam GUI illustrating real time streaming.

In an embodiment, the SAM application connects by Transmission Control Protocol (TCP) sockets to a Bluegiga iWRAP Access Server. The Bluegiga server may include a Linux computer, one Ethernet port, and three Bluetooth radios. These components together, for example with other aspects, form an embodiment of the SAM system SIU.

At startup, an embodiment of the SAM application uses User Datagram Protocol (UDP) to locate the Bluegiga server on the network. After connection to that Bluegiga server, the SAM application automatically attempts to connect wirelessly to the RSMs. If an RSM breaks its Bluetooth connection (e.g., by going out of range, losing battery power, etc.), the Bluegiga server removes the TCP socket and signals the SAM application that an RSM is “missing.” Wireless connection/reconnection may be an ongoing process, so that a returning RSM will be automatically routed to a free TCP port.

In an embodiment, the user can create, name, and configure “sessions” in order to group RSMs for display purposes. Up to 6 animals (or more or less as desired or required) can be selected for inclusion in each “session” (for example, up to 3 sessions—or more or less as desired or required—can be run simultaneously for a total of 18 RSMs). When each session is configured, the SAM application uses Universal Plug and Play (UPnP) discovery to learn the IP addresses of all available video cameras, and the user can select a camera to supply video for that session. A single camera can also be shared among several sessions.

In an embodiment, the SAM application offers, but not limited thereto, three selectable displays for each group of animals in a session: (1) the “Readouts” selection (FIG. 6) shows numeric displays for slow-speed data (reported at 1 Hz); (2) the “Plots” selection (FIG. 7) displays real-time waveforms (e.g., movement data, ECG, etc.) at up to 512 Hz; and (3) the “Video” selection shows the current image from the selected IP camera (this option would exist if a camera or the like is available). The real-time waveform transmission can be turned off to reduce radio usage and to save battery power. The user can also deselect an RSM (i.e., a specific animal) from the session, which will turn off all messaging and put the device in a low-power mode.

In an embodiment, the SAM application may use Java Database Connectivity (JDBC) to link to a Structured Query Language (SQL) database using the H2 Database Engine. The SQL database can store time-stamped data that is keyed to specific RSMs. Data collected by the SAM system can be selectively exported for specified time periods and/or specific animals into text files for analysis by other programs, such as Mathworks MATLAB or Microsoft Excel.

### Task 2: Evaluate SAM System in Dry Laboratory

Several special “evaluation” RSMs were created by the research team to facilitate the development of the SAM system and to debug the RSM embedded code. These RSMs were rapid prototypes that contained the same hardware as the final units, but no attempt in this instance was made to miniaturize them; the evaluation RSMs were used to verify electrical signals, to measure power consumption, and to provide a “back-door” communication channel for monitoring the devices during operation. After verifying the functionality of the evaluation units, the RSM embedded software was uploaded to the much smaller “production” RSM units.

The Bluetooth protocol was used for all wireless communications supported by the SAM system due to its high bandwidth capabilities and the wide availability of hardware and software support. The Bluegiga iWRAP Access Server (described above) was used to allow up to 18 RSMs (and hence 18 animals) to be connected simultaneously.

High-bandwidth wireless transmission evaluations were performed using a custom data-streaming protocol created by the research team. This protocol packs multiple samples of sensor data into a single data message (thus fully utilizing the Bluetooth bandwidth). This protocol allows data to be collected and streamed at rates up to 512 Hz.

A low-frequency data summary message protocol was also created to conserve bandwidth and power. This protocol provides RSM-computed averages for HR, RR, ST, activity, and battery status at a programmable rate ranging from 0.01 Hz to 1 Hz.

### Tasks 3 and 4: Test and Refine SAM System in Rats and Mice

For each experiment that was conducted as part of the instant effort by the present inventors, the animal was initially anesthetized in an induction chamber using 4-5% isoflurane anesthesia in oxygen. Each animal had a small portion of its upper torso shaved, and then had chemical depilatory cream applied to ensure that its fur did not interfere with the ECG electrodes. A SAM RSM (jacket and electronics) was then fitted to each animal, starting with the placement of the ECG electrodes, as described in Task 1.

### Initial Validation Using Rats

The SAM system was tested using one male and one female rat, weighing 350 g and 205 g, respectively. Each anesthetized animal was instrumented with both the SAM system and the Starr Life Sciences MouseOx Pulse Oximetry system (the criterion measurement system). The MouseOx non-invasive CollarClip sensor and proprietary software modules were used to collect data for the criterion measurement system.

Data were collected for ten minutes while the animal was supine and anesthetized. Each rat was then placed in a prone position and removed from the isoflurane mixture, allowing it to wake up. An additional ten minutes of data were collected as the animals regained consciousness and began to move around.

Note that although the rats were instrumented with both measurement systems (an embodiment of the SAM system and the current criterion MouseOx system), the presence of the current MouseOx components frequently became a substantial hindrance to motion, and the MouseOx signal degraded due to noise resulting from the animal's movements (such that it was often unusable as the rats regained consciousness).

### Initial Validation Using Mice

SAM system evaluations were also performed using one male and one female mouse, weighing 36.8 g and 28.8 g, respectively. As with the rats, the SAM and MouseOx systems were first tested while the mice were supine and anesthetized for ten minutes, and then while the animals were prone and conscious for an additional ten minutes.

Although the current MouseOx system was generally usable while the animals were anesthetized, the signal was completely corrupted by the movement of the animals during the second stage of the test; therefore, no comparison data are available for conscious mice due to failure of the criterion measurement system.

### Longer-Term Testing Using Multiple Rats and Mice Concurrently

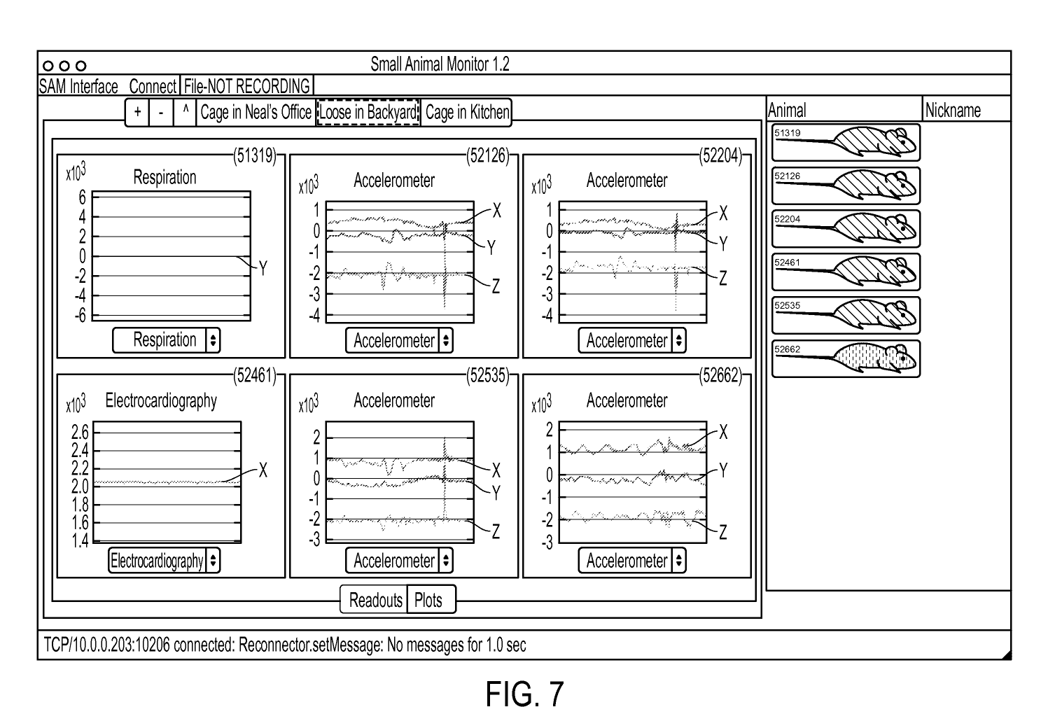

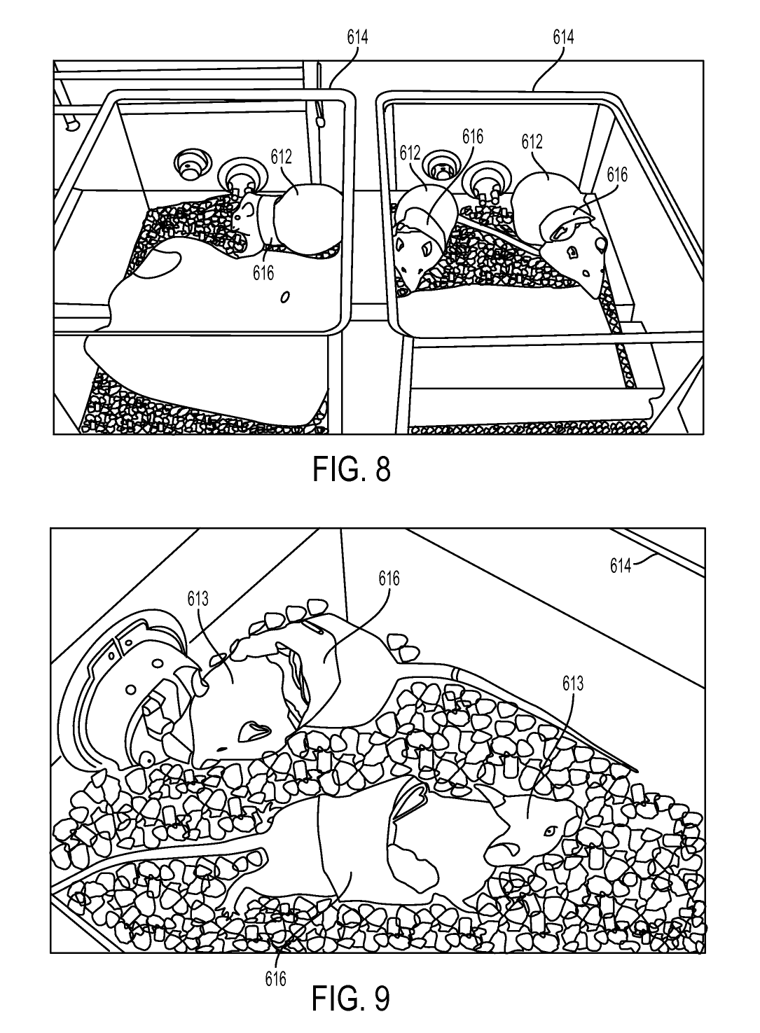

Once the individual animal testing was completed, multiple freely moving rats and mice were instrumented with an embodiment of the RSMs simultaneously. As shown in FIGS. 8 and 9, three rats 612 (FIG. 8) and two mice 613 (FIG. 9) were allowed to move freely while instrumented (all five animals were being monitored by the system simultaneously, though the rats where in separate containers from the mice). Data were collected for two and a half hours while the animals groomed and interacted with each other in their respective containers 614. For instance the rats and mice were awake while wearing an embodiment of the SAM system instrumentation.

The rats 612 were immediately comfortable with the RSM, moving around and exploring their surroundings. Although the mice 613 may have been slightly burdened by an embodiment of the RSM, they were still able move relatively easily around their container. None of the animals demonstrated an inclination to chew or perturb either their own RSM, or the RSM(s) worn by the other animal(s). The RSM jackets 616 were specially designed to be light and unobtrusive; their placement just behind the animals' front legs kept them from being able to reach or chew them (e.g., while grooming themselves).

### Task 5: Perform Data Analyses

Statistical analyses were performed on the HR and RR data collected concurrently by both the MouseOx and an embodiment of the present invention SAM systems. Note that skin temperature (collected by an embodiment of the SAM system) and core temperature (collected by the MouseOx system) are not directly comparable and are thus excluded from the statistical analyses.

During the periods when the animals regained consciousness and began to move around, the MouseOx system had frequent periods where the signals dropped out or were distorted based on the proprietary post-processing reports of the MouseOx software. These segments of data were excluded from the analyses; however manual measurements of HR and RR taken by the technicians performing the study determined that the observed values closely matched that of an embodiment of the present invention SAM-reported values.

The multi-animal trial did not use the MouseOx system, as all of the animals were conscious and mobile during this trial. The purpose of the multi-animal trial was to ensure that the system functioned appropriately while measuring multiple animals simultaneously, and to observe how the animals interacted with each other while instrumented. An embodiment of the present invention SAM system functioned as intended; summary data were reported at 1 Hz with no wireless data drop-outs for any of the animals. As mentioned, the animals were able to function with minimal impairment, and made no attempts to dislodge or chew their RSM or the RSM(s) of the other animals.

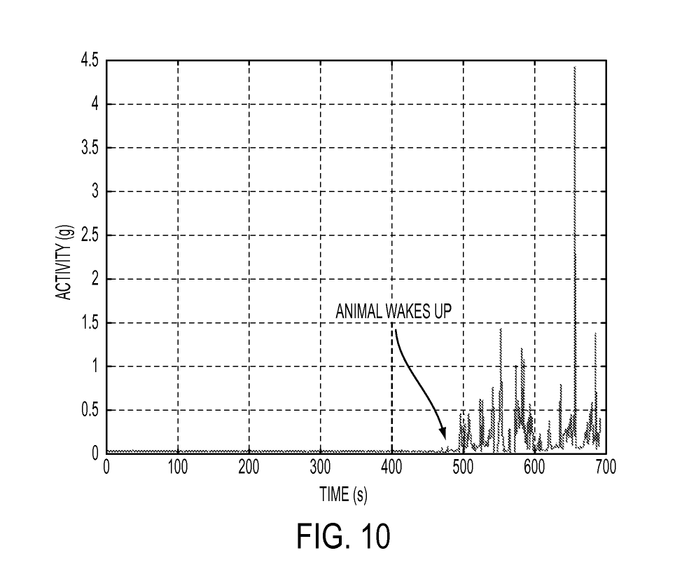

FIGS. 10-13 graphically depict example time series data streams collected using an embodiment of the present invention wireless SAM system on animals. In this example, activity data is calculated as an average acceleration magnitude over the sampling interval (e.g., 1 Hz).

### Statistical Analyses

The accuracy and precision of an embodiment of the SAM system in measuring HR and RR in relationship to the criterion MouseOx system were assessed via random-effects models. The rat and mouse data were analyzed separately. For each analysis, the animal's HR and RR measurements (in 15 s epochs) measured by the MouseOx system were subtracted from the animal's corresponding measurements determined by an embodiment of the SAM system and used as the response data. The random-effects model included a random intercept term that allowed the accuracy and the precision of the SAM system to vary from animal to animal. The parameters of the random-effects model were estimated by restricted maximum likelihood, and the 95% confidence interval for the mean within-subject measurement discrepancy was computed based on the t-distribution, whereas the 95% confidence interval for the within-subject variance component (i.e., standard deviation) was computed based on the standard normal distribution. The estimate for the mean within-subject measurement discrepancy and the estimate for the within-subject standard deviation were utilized to estimate the Bland Altman limits of measurement agreement. The MIXED procedure of SAS version 9.2 was used to conduct the analyses.

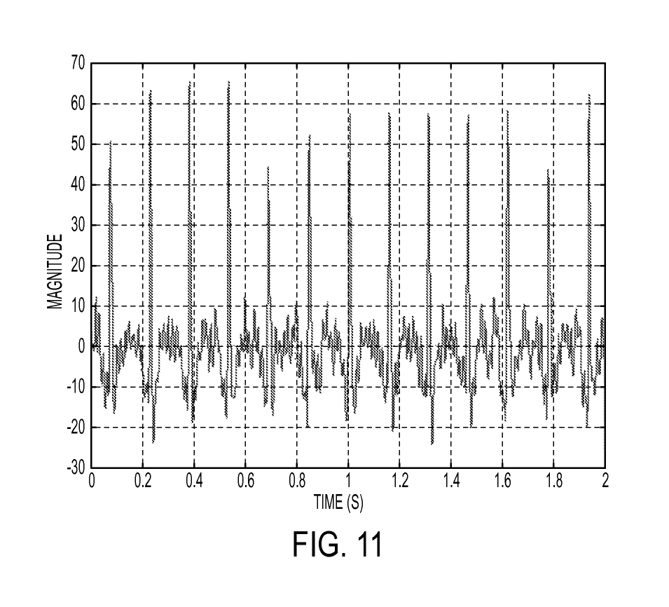

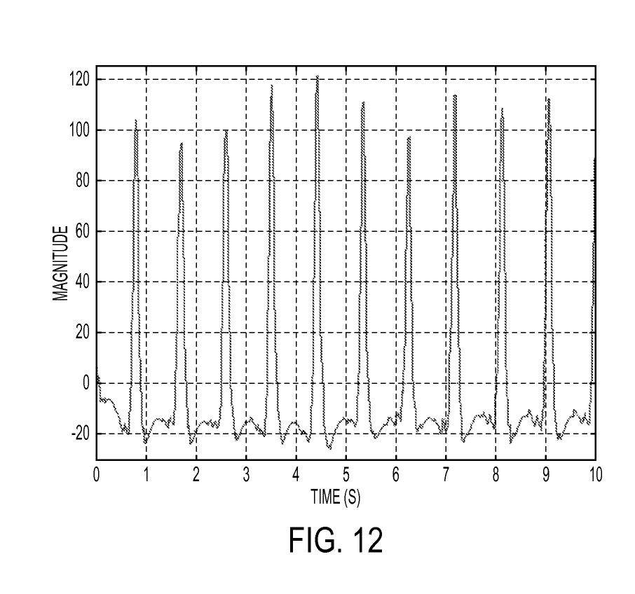

The HR and RR measurement discrepancies between the SAM and MouseOx systems for rats and mice are graphically summarized in FIGS. 14 and 15, respectively.

### Rat HR Measurement Agreement

The estimate for the mean HR measurement discrepancy between the two systems is −0.116 beats/min (95% CI: [−0.535, 0.302 beats/min], p=0.176), and the estimate for the within-subject measurement discrepancy standard deviation is 0.257 beats/min (95% CI: [0.222, 0.304 beat/min]). The estimate for the Bland Altman lower 95% limit of agreement is −0.630 beats/min (95% CI: [−0.724, −0.560 beats/minute]) and the estimate for the Bland Altman upper 95% limit of agreement is 0.390 beats/min (95% CI: [0.328, 0.492 beats/min]).

### Mouse HR Measurement Agreement

The estimate for the mean HR measurement discrepancy between the two systems is −0.859 beats/min (95% CI: [−4.782, 3.064 beats/min], p=0.220), and the estimate for the within-subject measurement discrepancy standard deviation is 1.273 beats/m (95% CI: [1.066, 1.583 beat/min]). The estimate for the Bland Altman lower 95% limit of agreement is −3.405 beats/minute (95% CI: [−4.025, −2.991 beats/min]) and the estimate for the Bland Altman upper 95% limit of agreement is 1.687 beats/min (95% CI: [1.273, 2.307 beats/min]).

### Rat RR Measurement Agreement

The estimate for the mean RR measurement discrepancy is −0.236 breaths/min (95% CI: [−14.560, 14.088 breaths/min], p=0.869), and the estimate for the within-subject measurement discrepancy standard deviation is 4.220 breaths/min (95% CI: [3.725, 4.869 breaths/min]). The estimate for the Bland Altman lower 95% limit of agreement is −8.676 breaths/min (95% CI: [−9.974, −7.686 breaths/min]) and the estimate for the Bland Altman upper 95% limit of agreement is 8.204 breaths/min (95% CI: [7.214, 9.502 breaths/min]).

### Mouse RR Measurement Agreement

The estimate for the mean RR measurement discrepancy is −1.707 breaths/min (95% CI: [−2.765, −0.648 breaths/min], p=0.002), and the estimate for the within-subject measurement discrepancy standard deviation is 3.309 breaths/min (95% CI: [2.711, 4.249 breaths/min]). The estimate for the Bland Altman lower 95% limit of agreement is −8.325 breaths/min (95% CI: [−10.205, −7.129 breaths/min]) and the estimate for the Bland Altman upper 95% limit of agreement is 4.911 breaths/min (95% CI: [3.715, 6.791 breaths/min]).

### Task 6: Perform Usability Study

A questionnaire was developed by the inventors that consisted of Likert-scale items and free-form responses. The questionnaire described the SAM system and its intended use, and was distributed to nine researchers throughout the United States who work with rodents.

A grant proposal stated that a prototype of the SAM system prototype would be considered “acceptable” if the average of all responses from all participants was above 4.0; the average of all respondents across all questions (shown below) was 4.3, thus surpassing the stated metric.

As shown in Table 1, the respondents were more interested in being able to monitor animals while awake and moving freely than anesthetized; this capability is noticeably lacking with current technology, as evidenced by the problems that were experienced with the criterion MouseOx system.

TABLE 1

| | Average |
|---|---|
| Question | Rating |
| A non-invasive device that could provide HR, RR, skin | 4.8 |
| temperature and a measure of activity in small animals | |
| would useful in my research. | |
| I would be interested in using such a device with | 2.7 |
| anesthetized rats. | |
| I would be interested in using such a device with | 4.6 |
| awake and freely moving rats. | |
| I would be interested in using such a device with | 3.3 |
| anesthetized mice. | |
| I would be interested in using such a device with | 4.4 |
| awake and freely moving mice. | |
| It would be beneficial to be able to monitor | 4.9 |
| multiple animals simultaneously. | |
| Remote monitoring would be important. | 4.1 |
| The ability to continuously monitor animals for up to | 4.5 |
| 3 hours is important. | |
| The ability to collect intermittent data over 24 | 4.8 |
| hours without changing the battery is important. | |
| The ability to set the interval time for collecting | 4.5 |
| data when recording intermittently is an important | |
| feature. | |
| I would be interested in testing a prototype of | 4.5 |
| the device. | |

For monitoring conscious animals, an embodiment of the SAM wireless system has an advantage over competing systems in that it does not introduce constraining wires or other tethers, nor does it require invasive surgery. Moreover, for example, other non-tethered systems require surgery on the subject or restrict the subject to a small platform or enclosure where measurements can be made. An embodiment of the SAM was able to record HR, RR, ST, and activity data on multiple awake, untethered animals. This capability is not available commercially and is desired by the research community. For example, one questionnaire respondent commented that he/she wanted to monitor the level of activity of multiple, untethered animals simultaneously post-surgery, a capability that is unique to the SAM system if the animals are to be allowed to interact (i.e., not caged separately). Another respondent cited the need for a system that can monitor mice reliably without impacting their movement or interaction with each other.

In an embodiment, the SAM system was able to successfully monitor multiple mice that were conscious and moving. The present inventors propose additional embodiments, including miniaturization to reduce the size of the circuit board, and creating a smaller, more ergonomic RSM jacket.

### Conclusions

An embodiment of the present invention wireless SAM system represents, but not limited thereto, a novel paradigm for noninvasively collecting physiologic and activity/motion/posture data on both rats and mice while either conscious or anesthetized. Using data collected on live animals in multiple experiments, an embodiment of the SAM system was shown to closely match the criterion MouseOx system, and in fact often exceeded the performance of the MouseOx system, particularly in conscious animals.

### Experimental Results and Examples Set No. 2

### Overview

An aspect of an embodiment of the present invention wireless SAM system may include, but not limited thereto, the following primary components: (1) the System Interface Unit (SIU) (or other processor or machine), which will function as a base station; (2) up to 18 or more Remote Sensor Modules (RSMs), which are data acquisition/transmission units that will be attached to the animals; (3) a PC (or other processor, system, device, or machine) and accompanying software, which will process, display, and store the received data; and (4) one or more optional wireless Internet Protocol (IP) cameras, which can record or display video in real time.

An embodiment of the RSMs may be self-contained units that house the circuitry required to, for example but not limited thereto, measure: (1) an ECG signal; (2) chest contraction and expansion; (3) motion activity of the animal (including body posture/orientation); (4) skin temperature; and (5) ambient air temperature. For example, in an embodiment, the RSM may rest on the dorsal surface of an animal (i.e., similar to a backpack) and will be held in place by flexible circuit board “straps” that will extend partially down both sides of the animal's torso, where each will attach to an ECG electrode (the straps will not extend the full circumference of the animal's torso).

In an embodiment, the straps may be thin flexible circuit boards that will convey the measured ECG voltage potentials to the data acquisition circuitry located on the dorsally-mounted motherboard. For example, the flexible circuit boards may have the consistency and thickness of plastic film. The straps may terminate with snaps, which will be physically connected to ECG electrodes affixed to the animal's torso; an accelerometer chip (used to measure respirations) may be incorporated into one of the straps. The dorsally-mounted backpack portion of the RSM will contain the radio frequency (RF) circuitry for communicating data wirelessly to the SIU. One of the backpack straps will include the RF antenna.

In an embodiment, the dorsally-mounted RSM may be enclosed in a small (3.5 cm×1.5 cm×0.5 cm) and lightweight (<10 g) plastic package (or other material) that it will fit on a mouse without impairing movement or other functions. In an embodiment, a lithium-polymer battery will power the RSM for at least 24 hours of continuous use (or a period as desired or required). The battery may be attached to the RSM via a custom connector that will allow it to be quickly and easily swapped out with a fully-charged battery, obviating the need to remove the entire RSM from the animal (e.g., for longer monitoring periods).

### System Interface Unit

In an embodiment, the SIU will serve as the coordinator for the wireless network and will be an aggregation point for data collected from deployed RSMs. The SIU may be based on a PC104 form factor single board computer, the TS-7200, manufactured by Technologic Systems [71]. In an embodiment, the SIU will also contain an RF daughter card, a wireless IEEE 802.11 (Wi-Fi) daughter card, and 18 or more charging cradles for the RSM batteries. Each SIU will be capable of reading telemetry information from up to 18 or more RSMs simultaneously.

In an embodiment, the RF PC104 daughter card may contain wireless circuitry for communicating with the RSMs, as well as processing capability for implementation of communication protocols. In addition to a standard (RJ-45) wired Ethernet connection, the SIU may be Wi-Fi compliant. This capability will enable the SIU to function similar to a standard wireless PC router; the SIU will be able to transmit data collected by the RSMs to remote PCs wirelessly. Additionally, Wi-Fi compliance may allow the SIU to aggregate data from other network capable devices (e.g., the optional IP camera(s)).

Other features that may be implemented regarding the SIU and RSM units, may include among other things the following: (1) a PC104 daughter card that will interface with the wireless RSM units; (2) a PC104 daughter card that will provide Wi-Fi capabilities; (3) software drivers for communication between the TS-7200's Linux operating system and the daughter cards; (4) a Linux-hosted application for providing TCP/IP access to system data via an IP over Ethernet connection (the Ethernet will be provided by the TS-7200); (5) an Interface Control Document that specifies the communication protocol that will enable the SIU to interface with PCs; (6) an output trigger signal that will allow third-party data collection systems to synchronize with the SAM system; and (7) mechanical housings associated with the SIU and RSM designs and the charging circuits for the swappable RSM battery.

### Remote Sensing Modules

An aspect of an embodiment of the present invention SAM system RSMs may contain, but not limited thereto, the following components: (1) a microcontroller; (2) a wireless transceiver; (3) flash memory; (4) two triaxial accelerometers (one dorsally-mounted in the backpack and the other in a backpack strap); (5) an ECG circuit; (6) two ECG electrodes; (7) two temperature sensors; and (8) a removable battery.

### Microcontroller

In an embodiment, the SAM backpack system RSMs may use the Texas Instruments CC430F5137 ultra low-power mixed-signal microcontroller, which has 32 KB of non-volatile Flash ROM (program memory) and 4 KB of volatile SRAM (scratchpad memory) [72]. This amount of memory is sufficient for storing the embedded on-line software code and provides the required run-time variable space to operate the unit.

The CC430F5137 has eight 12-bit analog-to-digital (A/D) converter inputs; three of the converters will receive 3-DOF activity data from the dorsally-mounted triaxial accelerometer chip at a rate of 32 Hz, a fourth may receive ECG data at a rate of 512 Hz, the fifth and sixth will receive the ST and AT, and the remaining two A/D converters will receive data from two of the three axes of the strap-mounted triaxial accelerometer chip. A universal communication interface may be used to pass cached data to an external memory chip (see below).

The CC430F5137 has five power-saving modes that are ideal for power-critical applications (e.g., a “standby” mode, which can be used between sampling instants, as the “wake-up” time from standby mode is less than 6 μs; in standby mode the current draw is only 1.6 μA).

The CC430F5137 contains the CC1101 RF transceiver core. The CC1101 is a sub-1 GHz transceiver designed for very-low-power wireless applications. The CC1101 is intended for the ISM (Industrial, Scientific and Medical) and SRD (Short Range Device) frequency bands at 315, 433, 868, and 915 MHz. The 915 MHz band may be used in the SAM prototype, for example.

### Memory

In an embodiment, each RSM may contain an 8 MB non-volatile Flash RAM chip, manufactured by Atmel, as a temporary cache for all collected data. The Flash RAM will allow the data to be sent in packets (rather than in a continuous stream) for optimal RF transmission.

### Accelerometers

In an embodiment, two Freescale Semiconductor MMA7260QT triaxial accelerometer modules will be used to monitor activity, chest movements, and body posture (i.e., orientation). One module may be mounted on the main circuit board of the RSM, which will reside on the dorsal aspect of the animal, and the second may be incorporated an appropriate distance along one of the strap flexible circuit boards (and possibly attached to the back of one of the strap female ECG snaps).

### Temperature Sensors

An aspect of an embodiment of the present invention prototype SAM system, ST and AT may be measured using two Measurement Specialties 10K3A1AM Thermistors configured in Wheatstone bridges. The skin temperature thermistor may contact the skin by being installed in one of the straps near an ECG electrode, and the ambient air temperature sensor will be exposed to the air at the cephalic end of the dorsally-mounted RSM backpack.

### Battery

In an embodiment, each SAM RSM may be powered by a 3.7 Vdc lithium polymer rectangular cell, the Renata Batteries Model 451730. The 451730 battery measures 3.06 cm×1.71 cm×0.47 cm and weighs 4.4 g. The battery in this instance may be the largest component in the RSM; it should be appreciated that smaller versions may be implemented. The 45173 battery is rated at 170 mAh and may last approximately 24 hours between charges. The battery can be completely recharged in less than two hours from any state of charge without negative impact. The SIU will contain 18 independent charging cradles (or more or less) for the RSM batteries, each of which will utilize built-in charging contacts (i.e., no wires).

### Electrodes

Example electrodes, may include, but not limited thereto, the 2670 3M Red Dot Repositionable Electrodes. Prior to application of the self-adhesive electrodes, each mouse or rat will be prepared by shaving and applying a depilatory to remove hair at the electrode sites. The electrodes may be modified to accommodate small mice and rats. If necessary to mechanically stabilize the caudal aspect of the RSM residing on the dorsal surface of the animal, a third, electrically-inoperative electrode will be placed on the dorsal surface of the animal and the abdominal end of the RSM will be designed to snap onto this electrode.

### Data Collection and Visualization Software

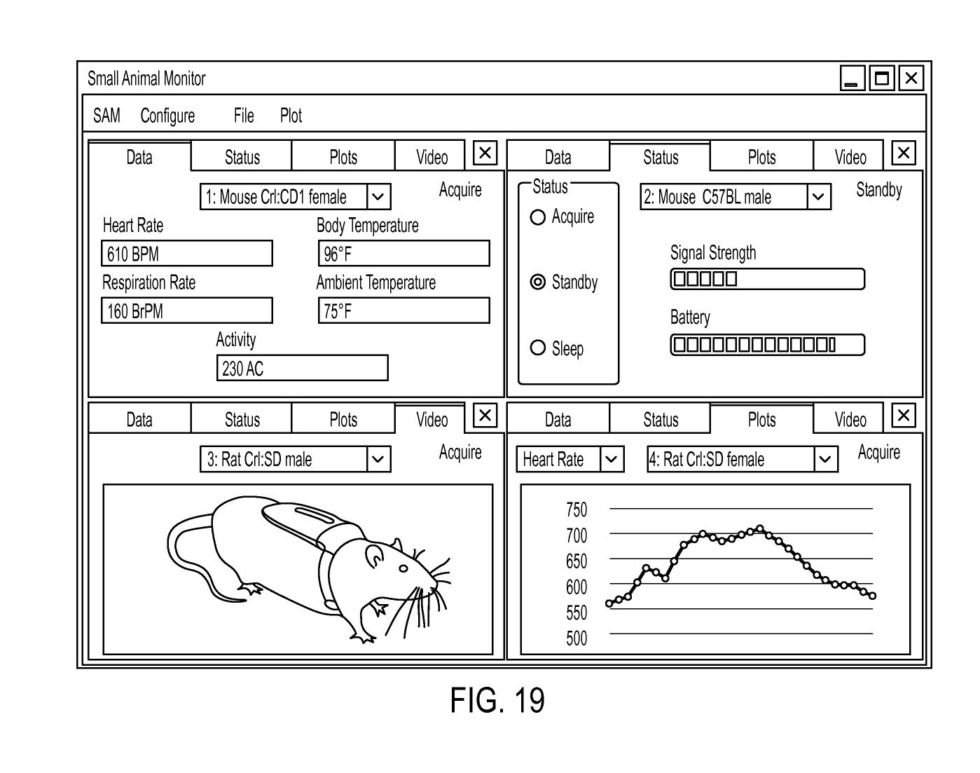

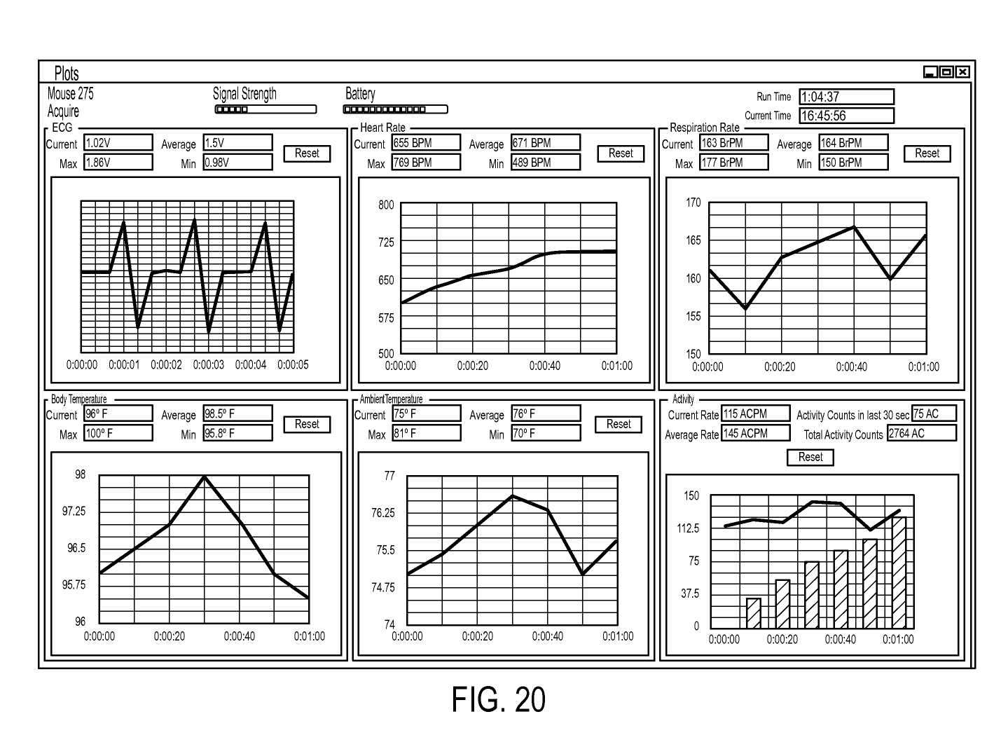

An aspect of an embodiment of the present invention SAM system may include a desktop software application that will be capable of simultaneously displaying the status of all 18 or more RSM data streams (or any subset thereof), including real-time access to each animal's physiological data and videographic monitoring with up to 18 or more cameras or other recordation devices. In an embodiment, the application may be written in Java (or other code) for portability across the major operating systems (e.g., Windows, Linux, and Mac OS X). Java's network interface package will provide socket connection from the desktop to the base station via Wi-Fi or wired Ethernet. For example, FIGS. 19 and 20 are illustrative screenshots of an embodiment of the SAM desktop software application.

For example, FIG. 19 shows the main display window (e.g., screenshot) of the SAM desktop software; the main window will be divided into subwindows, each corresponding to an RSM. In FIG. 19, four RSM windows are shown. Each RSM subwindow, which may be closed or resized, as desired, to focus on particular RSMs (i.e., particular animals), will have four “tabs” located at the top: Data, Status, Plots, and Video. Each tab, when selected via a mouse click, will display different information.

In an embodiment, the “Data” tab (shown in the upper-left RSM subwindow) may display various physiological data parameters (e.g., current HR, RR, etc.). The “Status” tab (shown in the upper-right RSM sub-window) may display the connection status of the RSM, including RF link quality and remaining battery power. The “Video” tab (which may not exist if there is no camera or the like) may show streaming video broadcast by the IP camera. The “Plots” tab (shown in the lower-right RSM subwindow) may show a graph of the currently selected (via a dropdown menu) physiologic parameter (e.g., heart rate) over time. The selected parameter may be changed at any time and the graph will update in real time. In an embodiment, to provide features such as zooming and panning through subsets of the plot data, the Java plot package from the Ptolemy II software system may be integrated with the SAM Java desktop application. The “Plot” menu selection may export the currently selected plot to a Joint Photographic Experts Group (JPEG) picture file for use in word-processed document or for printing.

When the graph located in the “Plots” tab is double-clicked, a new screen may be presented (e.g., screenshot of FIG. 20) which will show detailed information from one RSM, including the ability to graph multiple parameters simultaneously.

In an embodiment, the desktop software may archive all data in a SQL database. The “File” menu selection will provide navigation to the archival database, as well as selection of subsets of the data for playback. Data subsets will be available by animal, time range, and data type and will be implemented by SQL commands to the database via the Java Database Connectivity JDBC package.

### Video Camera

In an embodiment, the animal behavior may be monitored live, as well as video-recorded, via one or more wireless IP cameras (or other recordation devices) placed outside of the cage or wherever the researcher desires. The IP cameras may be closed-circuit television (CCTV) cameras that use a network protocol to transmit image data over the Internet or an internal Ethernet network. An embodiment of the SAM system's IP cameras may wirelessly interface with the SIU using its Wi-Fi capabilities; the video stream may be accessible, via the PC (or other machine, system, device or processor), for real-time display or stored to the PC's hard drive for archival. This functionality may be integrated into the PC software. A single WCS-2070 Wireless Day/Night IP Network Camera may be used in a prototype, however additional cameras may be added readily, as needed (each camera may be automatically assigned a unique IP address by the SIU).

### Wireless Transmission

In an embodiment, the wireless digital transmission protocol may be designed to allow a single SIU to coordinate to up to 18 or more RSMs (or less as desired or required) operating simultaneously on the same wireless network. In an embodiment, the protocol may be based on a Time Division Multiple Access (TDMA) methodology. In order to minimize the interference and facilitate Federal Communications Commission (FCC) approval an embodiment of the SAM system may use Frequency Hopping Spread Spectrum (FHSS), which is a method of transmitting radio signals by rapidly switching a carrier among many frequency channels, using a pseudorandom sequence known to both the transmitter and the receiver. Time slotting may be based on 7-slot frame, for example.

TDMA is a channel access method for shared medium networks. It allows several transmitters or transceivers to share the same frequency channel by dividing the signal into different time slots. The devices transmit in rapid succession, one after the other, each using its own time slot. This allows multiple devices to share the same transmission medium (e.g. radio frequency channel) while using only a part of its bandwidth.

Within the frame, one slot may be reserved for SIU transmission and 18 slots (or more if desired or required) may be reserved for RSM data transmission (each RSM may have a dedicated time slot). For example, each slot may be 6 ms in duration for a total frame time of 102 ms. At a proposed data rate of 250 kb/s, 6 ms provides for a maximum of 187 bytes to be transmitted per slot. Each slot transmission may contain approximately 16 bytes of header information (overhead), which will result in roughly 170 bytes of transmitted data payload from each RSM per slot and a theoretical throughput of 1.6 kB/s for each RSM.

In an embodiment, the required throughput per RSM, based on the sampling rates for the accelerometers, thermistors, and ECG circuitry data is approximately 1 kB/s. The theoretical throughput of 1.6 kB/s leaves 0.6 kB/s of bandwidth free to account for other factors, such as guard time and error detection.

The TDMA protocol is also efficient in terms of power; the duty cycle of each RSM will be less than 1/17^{th} for both transmission and reception. The actual duty cycle may be determined based on how the data are packetized, which provides an avenue for increasing battery life. For example, one possible technique for extending battery life would be to move HR processing to the RSM itself and only periodically perform (e.g., once per second) HR measurement, reporting a calculated HR value instead of streaming the ECG signal at 512 Hz for processing on the PC. All such options may be implemented, for example, as part of the present invention.

### SIU Discovery and Association

An aspect of an embodiment of the present invention RSM may begin scanning for an SIU-hosted network to join immediately upon power-up. During this scanning sequence, it will hop through the 900 MHz frequency spectrum, dwelling on each channel for a period of time, and listening for an SIU transmission. If the RSM receives an SIU transmission, the packet transmitted within the SIU slot may contain information regarding the hopping sequence the SIU is using and the present state of the SIU within the hopping sequence. This allows the RSM to determine where within the frequency spectrum the SIU will be transmitting on the next frame. At the end of the SIU's packet, it will transmit a “Clear To Send” message. This signals RSMs attempting to join the SIU network to transmit an association request to the SIU.

In an embodiment, the association request from the RSM may contain an identifier unique to the RSM along with information regarding its configuration. On the next frame, the SIU may transmit an association grant that the RSM will receive containing transmission information, such as which slot the RSM should use, along with channel and hopping sequence information.

Alternatively, in an embodiment, if the SIU presently has a full complement of 18 or more slaved RMSs, it may transmit a “Deny” message on the next frame. In response to the “Deny” message, the RSM will start scanning for another SIU with which to associate.

### RSM to SIU Pairing

In an embodiment, RSMs may have an “affinity” for a specific SIU. A command will be sent to each RSM instructing it to communicate only with a specific SIU. However, an RSM “discovery mode” may be implemented in the SIU in order to reverse the command, if necessary. In the discovery mode, the SIU will send out a command that will cause all unpaired RSMs to drop their affinity and associate with it, regardless of any prior pairing instructions. This functionality may allow an RSM channel that had been paired with a SIU, but which is no longer in operation, to be recovered.

### SIU Co-Existence

In an embodiment, the use of FHSS permits the operation of multiple SIU hosted networks in the same RF collision space, enabling greater than 18 animals (or more or less as desired or required) to be monitored using two or more SAM systems. The hopping sequence over the physical channel set may be determined by a pseudo-random sequence. Each SIU hosted network may be operating at a random offset in the sequence, hence multiple SIUs may operate in proximity without interfering with one another. The maximum number of SIUs that can operate in the same RF collision space is equal to the number of physical channels used in the hopping sequence.

### RSM Modes of Operation

In an embodiment, the RSMs may have three modes of operation (or more or if desired or required) to conserve battery power:

Sleep Mode. In sleep mode, for an embodiment, RSMs may go into the lowest power mode supported by their microcontrollers and circuitry. The RSM may wake from this mode on a periodic basis and scan for an SIU with which to associate. RSMs may automatically enter this mode if an SIU cannot be discovered in a specified period of time.

Standby Mode. In standby mode, for an embodiment, RSMs will be associated with an SIU, but may only transmit enough information to maintain the link. All signal processing circuits may be off to save power.

Active Mode. In active mode, for an embodiment, the RSMs will connect to an SIU and enable all sensor processing circuitry and full data transmission.

It should be appreciated that an aspect of system may include a computer system and the associated Internet connection upon which an embodiment may be implemented. Such configuration is typically used for computers (hosts) connected to the Internet and executing server or client (or a combination) software. A source computer such as laptop, an ultimate destination computer and relay servers, for example, as well as any computer or processor described herein, may use the computer system configuration and the Internet connection. An aspect of the system may be used as a portable electronic device such as a notebook/laptop computer, a media player (e.g., MP3 based or video player), a cellular phone, a Personal Digital Assistant (PDA), remote sensors, small animal physiological monitors (SAM), System Interface Unit (SIU), Graphical User Interface (GUI) an image processing device (e.g., a digital camera or video recorder), and/or any other handheld computing devices (e.g., a tablet PC), or a combination of any of these devices. Components of a computer system may vary, and it is not intended to be limited to any particular architecture or manner of interconnecting the components; as such details are not germane to the present invention. It will also be appreciated that network computers, handheld computers, cell phones and other data processing systems which have fewer components or perhaps more components may also be used. An aspect of computer system may, for example, be an Apple Macintosh computer or Power Book, or an IBM compatible PC. An aspect of a computer system may includes a bus, an interconnect, or other communication mechanism for communicating information, and a processor, commonly in the form of an integrated circuit, coupled with bus for processing information and for executing the computer executable instructions. An aspect of a computer system may also includes a main memory, such as a Random Access Memory (RAM) or other dynamic storage device, coupled to bus for storing information and instructions to be executed by the processor.

Main memory also may be used for storing temporary variables or other intermediate information during execution of instructions to be executed by the processor. An aspect of a computer system may further include a Read Only Memory (ROM) (or other non-volatile memory) or other static storage device coupled to bus for storing static information and instructions for processor. A storage device, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, may be coupled to the bus for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. An aspect of a computer system may include an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include, for example, any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

A computer system may be coupled via bus to a display, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device, including alphanumeric and other keys, is coupled to bus for communicating information and command selections to processor. Another type of user input device is cursor control, such as a mouse, a trackball, or cursor direction keys for communicating direction information and command selections to processor and for controlling cursor movement on display. This input device typically has two degrees of freedom in two axes, a first axis (e.g., x) and a second axis (e.g., y), that allows the device to specify positions in a plane.

A computer system may be used for implementing the methods and techniques described herein. According to one embodiment, those methods and techniques are performed by the computer system in response to the processor executing one or more sequences of one or more instructions contained in main memory. Such instructions may be read into main memory from another computer-readable medium, such as storage device. Execution of the sequences of instructions contained in main memory causes processor to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus. Transmission media can also take the form of acoustic or light waves, such as those generated during radio-wave and infrared data communications, or other form of propagated signals (e.g., carrier waves, infrared signals, digital signals, etc.). Transmission media may be in wireless format. Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor for execution. For example, the instructions may initially be carried on a magnetic disk of a remote computer. The remote computer can load the instructions into its dynamic memory and send the instructions over a telephone line using a modem. A modem local to computer system can receive the data on the telephone line and use an infrared transmitter to convert the data to an infrared signal. An infrared detector can receive the data carried in the infrared signal and appropriate circuitry can place the data on bus. A bus carries the data to main memory, from which processor retrieves and executes the instructions. The instructions received by main memory may optionally be stored on storage device either before or after execution by processor.

A computer system may also include a communication interface coupled to bus. Communication interface may provide a two-way data communication coupling to a network link that is connected to a local network. For example, communication interface may be an Integrated Services Digital Network (ISDN) card or a modem to provide a data communication connection to a corresponding type of telephone line. As another non-limiting example, communication interface may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. In any such implementation, communication interface sends and receives electrical, electromagnetic or optical signals that carry digital data streams representing various types of information.

Network link typically provides data communication through one or more networks to other data devices. For example, a network link may provide a connection through local network to a host computer or to data equipment operated by an Internet Service Provider (ISP) 142. ISP in turn provides data communication services through the worldwide packet data communication network Internet. Local network and Internet may both use electrical, electromagnetic or optical signals that carry digital data streams. The signals through the various networks and the signals on the network link and through the communication interface, which carry the digital data to and from a computer system, are exemplary forms of carrier waves transporting the information.

A received code may be executed by the processor as it is received, and/or stored in storage device, or other non-volatile storage for later execution. In this manner, computer system may obtain application code in the form of a carrier wave.

The concept of real-time wireless non-invasive animal monitoring system and related method, among other things, has been developed by the inventors. As disclosed herein, an aspect of an embodiment provides, among other things, the capability to monitor up to a plurality of animals (or subjects) simultaneously, with continuous recording, synchronization, and display of physiological data, including heart rate, respiratory rate, motion/posture activity, skin temperature, and ambient temperature; and may be implemented and utilized with the related processors, networks, computer systems, internet, and components and functions according to the schemes disclosed herein.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient/subject or machine/system/computer/processor. Moreover, the various components may be in communication via wireless and/or hardwire or other desirable and available communication means, systems and hardware. Moreover, various components and modules may be substituted with other modules or components that provide similar functions.

### ADDITIONAL EXAMPLES

### Example 1

An aspect of an embodiment of the present invention provides, but not limited thereto, a system for monitoring a subject. The system may comprise: at least one sensor module disposed in communication with the subject and configured to obtain data from the subject; at least one processor module configured to receive the subject data; a transmission module or transceiver module configured to transmit the subject data, wherein the transmission comprises wireless transmission to an output module that is remote relative to the subject; and wherein at least one of the at least one sensor module, the at least one processor module, or the transmission module is configured to be disposed in communication with a subject wearable interface.

### Example 2

The system of example 1, further comprising a power source in communication with the monitoring system.

### Example 3

The system of example 2, wherein the power source is configured to be disposed in communication with the subject wearable interface.

### Example 4

The system of example 1 (as well as subject matter of one or more of any combination of examples 2-3), wherein the subject data comprises physiological data of the subject.

### Example 5

The system of example 4 (as well as subject matter of one or more of any combination of examples 2-3), wherein the physiological data is derived from an image recording system.

### Example 6

The system of example 5 (as well as subject matter of one or more of any combination of examples 2-4), wherein the image recording system obtains visible wavelengths, infrared wavelengths, ultraviolet wavelengths, or X-ray wavelengths.

### Example 7

The system of example 4 (as well as subject matter of one or more of any combination of examples 2-3 or 5-6), wherein the physiological data includes any one or more of the following: heart rate (HR) data, respiratory rate (RR) data, ECG data, EEG data, arterial oxygen saturation (SaO2), photoplethysmography data, temperature data, or chest contraction and expansion data.

### Example 8

The system of example 1 (as well as subject matter of one or more of any combination of examples 2-7), wherein the subject data comprises one or more of any combination of the following: ECG data, heart rate (HR) data, chest contraction and expansion data, inertial forces data imposed by the subject or on the subject by gravity, or temperature data at the subject.

### Example 9

The system of example 8 (as well as subject matter of one or more of any combination of examples 2-7), wherein the processor module may be configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

### Example 10

The system of example 1 (as well as subject matter of one or more of any combination of examples 2-9), wherein the subject is an animal.

### Example 11

The system of example 10 (as well as subject matter of one or more of any combination of examples 2-9), wherein the animal is a rodent.

### Example 12

The system of example 1 (as well as subject matter of one or more of any combination of examples 2-11), further comprising a subject wearable interface configured for accommodating one or more of any combination of the following: the at least one sensor module, the processor module, the wireless communication module, and a power source.

### Example 13

The system of example 12 (as well as subject matter of one or more of any combination of examples 2-11), further comprising a power source in communication with the monitoring system.

### Example 14

The system of example 1 (as well as subject matter of one or more of any combination of examples 2-13), wherein the remote output module comprises one or more of any combination of the following: storage, memory, network, or display.

### Example 15

The system of example 1 (as well as subject matter of one or more of any combination of examples 2-14), wherein the transmission further comprises: hard-wired transmission or wireless transmission to an output module that is local to the subject.

### Example 16

The system of example 15 (as well as subject matter of one or more of any combination of examples 2-14), wherein the local output module is configured to be disposed in communication with the subject wearable interface.

### Example 17

The system of example 15 (as well as subject matter of one or more of any combination of examples 2-14 or 16), wherein the local output module comprises one or more of any combination of: storage or memory.

### Example 18

An aspect of an embodiment of the present invention provides, but not limited thereto, a method for monitoring a subject. The method may comprise: providing a subject wearable interface; disposing at least one sensor module in communication with the subject, wherein the at least one sensor module is configured to be in communication with the subject wearable interface; obtaining data from the subject using the at least one sensor module; and transmitting the subject data to an output module that is remote relative to the subject.

### Example 19

The method of example 18, wherein the subject data comprises physiological data of the subject.

### Example 20

The method of example 19, wherein the physiological data is derived from imaging the subject.

### Example 21

The method of example 20, wherein the imaging obtains visible wavelengths, infrared wavelengths, ultraviolet wavelengths, or X-ray wavelengths.

### Example 22

The method of example 19 (as well as subject matter of one or more of any combination of examples 20-21), wherein the physiological data includes any one or more of the following: heart rate (HR) data, respiratory rate (RR) data, ECG data, EEG data, arterial oxygen saturation (SaO2), photoplethysmography data, temperature data, or chest contraction and expansion data.

### Example 23

The method of example 18 (as well as subject matter of one or more of any combination of examples 19-22), wherein the subject data comprises one or more of any combination of the following: ECG data, heart rate (HR) data, chest contraction and expansion data, inertial forces data imposed by the subject or on the subject by gravity, or temperature data at the subject.

### Example 24

The method of example 23 (as well as subject matter of one or more of any combination of examples 19-22), wherein the processor module may be configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

### Example 25

The method of example 18 (as well as subject matter of one or more of any combination of examples 19-24), wherein the subject is an animal.

### Example 26

The method of example 25 (as well as subject matter of one or more of any combination of examples 19-24), wherein the animal is a rodent.

### Example 27

The method of example 18 (as well as subject matter of one or more of any combination of examples 19-26), wherein the remote output module comprises one or more of any combination of the following: storage, memory, network, or display.

### Example 28

The method of example 18 (as well as subject matter of one or more of any combination of examples 19-27), wherein the transmitting further comprises: hard-wired transmitting or wireless transmitting to an output module that is local to the subject.

### Example 29

The method of example 28 (as well as subject matter of one or more of any combination of examples 19-27), further comprises disposing the local output module in communication with the subject wearable interface.

### Example 30

The method of example 28 (as well as subject matter of one or more of any combination of examples 19-27 or 29), wherein the local output module comprises one or more of any combination of: storage or memory.

### Example 31

An aspect of an embodiment of the present invention provides, but not limited thereto, a non-transitory machine readable medium including instructions, which when executed by a machine, cause the machine to: obtain data from the subject using at least one sensor module; and transmit the subject data to an output module that is remote relative to the subject.

### Example 32

The non-transitory machine readable medium of example 31, wherein the subject data comprises physiological data of the subject.

### Example 33

The non-transitory machine readable medium of example 32, wherein the physiological data is derived from imaging the subject.

### Example 34

The non-transitory machine readable medium of example 33, wherein the imaging obtains visible wavelengths, infrared wavelengths, ultraviolet wavelengths, or X-ray wavelengths.

### Example 35

The non-transitory machine readable medium of example 32 (as well as subject matter of one or more of any combination of examples 33-34), wherein the physiological data includes any one or more of the following: heart rate (HR) data, respiratory rate (RR) data, ECG data, EEG data, arterial oxygen saturation (SaO2), photoplethysmography data, temperature data, or chest contraction and expansion data.

### Example 36

The non-transitory machine readable medium of example 31 (as well as subject matter of one or more of any combination of examples 32-35), wherein the subject data comprises one or more of any combination of the following: ECG data, heart rate (HR) data, chest contraction and expansion data, inertial forces data imposed by the subject or on the subject by gravity, or temperature data at the subject.

### Example 37

The non-transitory machine readable medium of example 36 (as well as subject matter of one or more of any combination of examples 32-35), further configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

### Example 38

The non-transitory machine readable medium of example 31 (as well as subject matter of one or more of any combination of examples 32-37), wherein the subject is an animal.

### Example 39

The non-transitory machine readable medium of example 38 (as well as subject matter of one or more of any combination of examples 32-37), wherein the animal is a rodent.

### Example 40

The non-transitory machine readable medium of example 31 (as well as subject matter of one or more of any combination of examples 32-39), wherein the remote output module comprises one or more of any combination of the following: storage, memory, network, or display.

### Example 41

The non-transitory machine readable medium of example 31 (as well as subject matter of one or more of any combination of examples 32-40), wherein the transmitting further comprises: hard-wired transmitting or wireless transmitting to an output module that is local to the subject.

### Example 42

The non-transitory machine readable medium of example 41 (as well as subject matter of one or more of any combination of examples 32-40), wherein the local output module comprises one or more of any combination of: storage or memory.

### Example 43

The method of using any of the systems or its components provided in any one or more of examples 1-18.

### Example 44

The method of manufacturing any of the systems or its components provided in any one or more of examples 1-18.

### Example 45

A non-transitory machine readable medium including instructions for monitoring a subject, which when executed by a machine, cause the machine to perform any of the steps or activities provided in any one or more of examples 18-30.

### Example 46

A non-transitory computer readable medium including program instructions for monitoring a subject, wherein execution of the program instructions by one or more processors of a computer system causes the processor to carry out: any of the steps or activities provided in any one or more of examples 18-30.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A system for monitoring a subject, said system comprising:
at least one sensor module disposed in communication with the subject and configured to obtain data from the subject;
at least one processor module configured to receive said subject data;
a transmission module or transceiver module configured to transmit said subject data, wherein said transmission comprises wireless transmission to an output module that is remote relative to the subject; and
wherein at least one of said at least one sensor module, said at least one processor module, or said transmission module is configured to be disposed in communication with a subject wearable interface.

2. The system of claim 1, further comprising a power source in communication with said monitoring system.

3. The system of claim 2, wherein said power source is configured to be disposed in communication with said subject wearable interface.

4. The system of claim 1, wherein said subject data comprises physiological data of the subject.

5. The system of claim 4, wherein said physiological data is derived from an image recording system.

6. The system of claim 5, wherein said image recording system obtains visible wavelengths, infrared wavelengths, ultraviolet wavelengths, or X-ray wavelengths.

7. The system of claim 4, wherein said physiological data includes any one or more of the following: heart rate (HR) data, respiratory rate (RR) data, ECG data, EEG data, arterial oxygen saturation (SaO2), photoplethysmography data, temperature data, or chest contraction and expansion data.

8. The system of claim 1, wherein said subject data comprises one or more of any combination of the following: ECG data, heart rate (HR) data, chest contraction and expansion data, inertial forces data imposed by the subject or on the subject by gravity, or temperature data at the subject.

9. The system of claim 8, wherein said processor module may be configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

10. The system of claim 1, wherein the subject is an animal.

11. The system of claim 10, wherein the animal is a rodent.

12. The system of claim 1, further comprising a subject wearable interface configured for accommodating one or more of any combination of the following: said at least one sensor module, said processor module, said wireless communication module, and a power source.

13. The system of claim 12, further comprising a power source in communication with said monitoring system.

14. The system of claim 1, wherein said remote output module comprises one or more of any combination of the following: storage, memory, network, or display.

15. The system of claim 1, wherein said transmission further comprises: hard-wired transmission or wireless transmission to an output module that is local to the subject.

16. The system of claim 15, wherein said local output module is configured to be disposed in communication with said subject wearable interface.

17. The system of claim 15, wherein said local output module comprises one or more of any combination of: storage or memory.

18. A method for monitoring a subject, said method comprising:
providing a subject wearable interface;
disposing at least one sensor module in communication with the subject, wherein said at least one sensor module is configured to be in communication with said subject wearable interface;
obtaining data from the subject using said at least one sensor module; and
transmitting said subject data to an output module that is remote relative to the subject.

19. The method of claim 18, wherein said subject data comprises physiological data of the subject.

20. The method of claim 19, wherein said physiological data is derived from imaging the subject.

21. The method of claim 20, wherein said imaging obtains visible wavelengths, infrared wavelengths, ultraviolet wavelengths, or X-ray wavelengths.

22. The method of claim 19, wherein said physiological data includes any one or more of the following: heart rate (HR) data, respiratory rate (RR) data, ECG data, EEG data, arterial oxygen saturation (SaO2), photoplethysmography data, temperature data, or chest contraction and expansion data.

23. The method of claim 18, wherein said subject data comprises one or more of any combination of the following: ECG data, heart rate (HR) data, chest contraction and expansion data, inertial forces data imposed by the subject or on the subject by gravity, or temperature data at the subject.

24. The method of claim 23, wherein said processor module may be configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

25. The method of claim 18, wherein the subject is an animal.

26. The method of claim 25, wherein the animal is a rodent.

27. The method of claim 18, wherein said remote output module comprises one or more of any combination of the following: storage, memory, network, or display.

28. The method of claim 18, wherein said transmitting further comprises: hard-wired transmitting or wireless transmitting to an output module that is local to the subject.

29. The method of claim 28, further comprises disposing said local output module in communication with said subject wearable interface.

30. The method of claim 28, wherein said local output module comprises one or more of any combination of: storage or memory.

31. A non-transitory machine readable medium including instructions, which when executed by a machine, cause the machine to:
obtain data from the subject using at least one sensor module; and
transmit said subject data to an output module that is remote relative to the subject.

32. The non-transitory machine readable medium of claim 31, wherein the subject data comprises physiological data of the subject.

33. The non-transitory machine readable medium of claim 32, wherein said physiological data is derived from imaging the subject.

34. The non-transitory machine readable medium of claim 33, wherein said imaging obtains visible wavelengths, infrared wavelengths, ultraviolet wavelengths, or X-ray wavelengths.

35. The non-transitory machine readable medium of claim 32, wherein said physiological data includes any one or more of the following: heart rate (HR) data, respiratory rate (RR) data, ECG data, EEG data, arterial oxygen saturation (SaO2), photoplethysmography data, temperature data, or chest contraction and expansion data.

36. The non-transitory machine readable medium of claim 31, wherein said subject data comprises one or more of any combination of the following: ECG data, heart rate (HR) data, chest contraction and expansion data, inertial forces data imposed by the subject or on the subject by gravity, or temperature data at the subject.

37. The non-transitory machine readable medium of claim 36, further configured to provide any one or more of the following: a) compute heart rate (HR) data derived from ECG signal data; b) compute respiratory rate (RR) derived from the chest contraction and expansion data; c) compute movements of the subject or gravitational forces imposed on the subject derived from the accelerometry data; d) skin temperature (ST) derived from the temperature data at the subject; e) computing heart rate (HR) from photoplethysmography data or sensor; or f) inferring respiration from a heart rate (HR) signal.

38. The non-transitory machine readable medium of claim 31, wherein the subject is an animal.

39. The non-transitory machine readable medium of claim 38, wherein the animal is a rodent.

40. The non-transitory machine readable medium of claim 31, wherein said remote output module comprises one or more of any combination of the following: storage, memory, network, or display.

41. The non-transitory machine readable medium of claim 31, wherein said transmitting further comprises:
hard-wired transmitting or wireless transmitting to an output module that is local to the subject.

42. The non-transitory machine readable medium of claim 41, wherein said local output module comprises one or more of any combination of: storage or memory.
